(19) United States
(12) Patent Application Publication
(10) Pub. No.: US 2025/0295722 A1
(43) Pub. Date: Sep. 25, 2025

(54) METHOD FOR THE PRODUCTION OF A PLANT EXTRACT

(71) Applicant: Vertanical GmbH, Gräfelfing (DE)

(72) Inventors: Bastian Baasch, Utting am Ammersee (DE); Clemens Fischer, München (DE)

(21) Appl. No.: 18/833,550

(22) PCT Filed: Jan. 27, 2023
(86) PCT No.: PCT/EP2023/052073

§ 371 Date: Jul. 26, 2024

(30) Foreign Application Priority Data
Jan. 28, 2022 (EP) 22 154 007.3

(51) Int. Cl. A61K 36/185; A61K 31/00

(52) U.S. Cl. CPC A61K 36/3482; A61K 31/658; A61K 2236/15; A61K 2236/17; A61K 2236/35

(57) ABSTRACT

The present invention generally relates to methods for producing (a) plant extract(s), preferably a Cannabis plant extract. In particular, the present invention relates to a method for producing a Cannabis plant extract comprising delta-9-tetrahydrocannabinol (THC) from a Cannabis plant comprising the following steps: (a) providing a Cannabis plant which comprises delta-9-tetrahydrocannabinol (THC) in a sufficient amount: (b) trimming and drying the flower material separated from the remaining plant material; and (c) treating the flower material of step (b) with a solvent and separating the Cannabis plant extract comprising delta-9-tetrahydrocannabinol (THC) from the flower material. Furthermore, the invention relates to a Cannabis plant extract comprising delta-9-tetrahydrocannabinol (THC) as produced by/obtainable by the methods described herein. Furthermore, the invention relates to the Cannabis plant extract as produced by/obtainable by the herein described methods for use in medicine. Moreover, the present invention relates to the Cannabis plant extract as produced by/obtainable by the herein described methods for use in the treatment and/or prevention of chronic cancer pain, somatic pain, visceral pain, central neuropathic pain, peripheral neuropathic pain and complex pain syndromes.

### CROSS-REFERENCE TO RELATED APPLICATIONS

This application is a National Stage Entry under 35 U.S.C. § 371 of International Application No. PCT/EP2023/052073, filed on Jan. 27, 2023 and published on Aug. 3, 2023 under Publication No. WO 2023/144340, which claims the benefit of European Patent Application No. 22154007.3, filed on Jan. 28, 2022, the entireties of each of which are herein incorporated herein by reference.

### FIELD OF INVENTION

The present invention generally relates to methods for producing (a) plant extract(s), preferably a Cannabis plant extract. In particular, the present invention relates to a method for producing a Cannabis plant extract comprising delta-9-tetrahydrocannabinol (THC) from a Cannabis plant comprising the following steps:
(a) providing a Cannabis plant which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract comprising delta-9-tetrahydrocannabinol (THC) from the flower material.

Furthermore, the invention relates to a Cannabis plant extract comprising delta-9-tetrahydrocannabinol (THC) as produced by/obtainable by the methods described herein. Furthermore, the invention relates to the Cannabis plant extract as produced by/obtainable by the herein described methods for use in medicine. Moreover, the present invention relates to the Cannabis plant extract as produced by/obtainable by the herein described methods for use in the treatment and/or prevention of chronic cancer pain, somatic pain, visceral pain, central neuropathic pain, peripheral neuropathic pain or complex pain syndromes.

### BACKGROUND OF INVENTION

Cannabis has been known as an herbal medicine for a long time. However only since the 19th century it has been investigated and used more systematically in medicine. Not much later, in the early 20th century, Cannabis research was however mostly discontinued again, as its psychoactive effect and its resulting abuse were deemed to outweigh its benefits as a source of medicine. Today, Cannabis is still considered an illegal drug in most jurisdictions and therefore is subject to extensive legislative regulation. Only recently, the aforementioned legislative regulations were partially alleviated in some regions of this world, in view of an altered perspective of its risk profile as an abusive drug.

The main active substances rendering Cannabis as an herbal medicine relevant are cannabinoids. Cannabinoids are a substance class derived from Cannabis plants. One of the most relevant cannabinoids is the psychoactive delta-9-tetrahydrocannabinol (THC). However, until now more than 100 cannabinoids have been identified in Cannabis plants, many of them lacking psychoactive activity. Most of these cannabinoids are—similar to THC—only poorly soluble in water, but well soluble in organic solvents, such as hydrocarbons and alcohols. Many cannabinoids, particularly the mixture of cannabinoids with further substances found in Cannabis plants, are subject to research in several medical indications, including but not limited to neuropathic pain, fibromyalgia, rheumatoid arthritis, and mixed chronic pain (Bridgeman, M. B. & Abazia, D. T., “Medical Cannabis: History, Pharmacology, and implications for the acute care setting”, P&T 42(3), 2007, pp. 180-188).

More specifically, plant-derived Cannabis medicinal extracts were found to be superior to placebo in relieving pain in patients with multiple sclerosis, spinal cord injury, brachial plexus damage and limp amputation due to neurofibromatosis. It was found that both THC and cannabidiol (CBD) alone as well as in a 1:1 CBD: THC ratio were effective (Wade, D. T. et al., Clin. Rehabil. 2003, 17(1), pp. 21-29).

Similar results were obtained in using a whole plant extract in comparison to THC and placebo, while a slight, statistically not significant benefit of the whole plant extract over THC was observed in multiple sclerosis patients for treating pain and spasms (Zajicek J et al., Lancet. 2003, 362(9395), pp. 1517-1526).

In consequence, further research was dedicated to the complex interaction of the several cannabinoids and other substances in whole plant extracts of Cannabis, resulting in the finding that CBD does exert an antinociceptive and antihyperalgesic effect to result in pain relief in rat models. Within that more mechanistically oriented study, it was argued that the further constituents of the extract, such as terpenes and flavonoids may synergistically contribute to such activity of CBD. Also the pain relief effect of Cannabis was found to be induced not only by its antinociceptive, but also by an anti-inflammatory activity (Comelli F. et al., Phytotherapy Research 2008, 22(8), pp. 1017-1024).

Thus, both the anti-inflammatory activity as well as the complex interplay between the main cannabinoids (such as THC—in the form of its acid tetrahydrocannabinolic acid (THC (A)) and cannabidiol (CBD)) and other constituents of Cannabis extracts were further investigated. This resulted in the finding, that THC (A) is deemed to be the main anti-inflammatory active at higher doses, while CBD has anti-inflammatory activity only at lower dosages, but cytotoxic activity at higher doses. It was further found in cell culture models using HCT116 and CaCO2 cells that Cannabis extracts are even more active in reducing inflammation than the single cannabinoids alone, while there is already a difference in activity when comparing an extract from fresh flower to baked flowers of Cannabis. The Cannabis extracts were fractionated, but there was no dedicated finding on which individual compounds in the fractions actually contributed to the main activities attributed to either CBD and/or THC (A) (Nallathambi R. et al., Cannabis and Cannabinoid Research 2017, 2(1), pp. 167-182).

Today, there is no doubt that these cytotoxic (and thus potentially relevant to cancer-treatment) and anti-inflammatory activities are substantially derived not only from the main constituents THC (A) and/or CBD. There is also a contribution from the aforementioned terpenes, which however substantially deviate from specific variety to variety of the Cannabis plant (Gallily R. et al., Cannabis and Cannbinoid Research 2018, 3(1), pp. 282-290). In particular, it has been established from plant physiology, that any Cannabis plant that preferably comprises CBD can be discriminated from a variety that comprises primarily THC (A) also on the basis of associated patterns of terpenes. Therefore, certain cytotoxic and potentially also other effects (such anti-inflammatory or antinociceptive/antihyperalgesic effects) mainly derived from CBD and/or THC (A) are each influenced by a specific set of terpenes associated with such main cannabinoid. As a matter of example, CBD is active as a cytotoxic agent and is associated with the terpenes guaiol and alpha-bisabolol and it was found that only in combination with the dominant phytocannabinoid those terpenes exert activity, while the activity of the main phytocannabinoid can only be increased by those specific terpenes. This is termed the “inter-entourage effect” of phytocannabinoids (Namdar D. et al., Molecules 2019, 24(3031), pp. 1-17).

Therefore, research has been dedicated to processes for extraction of pharmaceutically active substances from Cannabis plants. Desired is a maximum yield of the cannabinoids, and optionally terpenes, terpenoids and/or flavonoids. Furthermore, it is beneficial that the relative concentrations of said pharmaceutically active substances is maintained to preserve potential synergism between the substances when applied as a pharmaceutical drug. At the same time, it is usually desired that solids and waxes are to be removed, to obtain a pure extract that can be subjected to further purification and/or chemical treatment. Several Cannabis plant extracts and corresponding uses have been described but not necessarily with specific production methods (e.g. WO 2020/006598 and WO 2020/006599)

The above objective is further constrained by general, environmental and economical boundary conditions. For example, it is beneficial to minimize the amounts of (organic) solvents and/or energy during preparation of the extract.

Accordingly, sophisticated separation technologies have been evaluated employing supercritical CO₂ as a solvent which can be removed from the final product with no (pharmaceutically) relevant residuals (described in e.g. WO 2002/064109 A2). DE 103 37 458 A1 discloses processes for the extraction of pharmaceutically active substance mixtures from Cannabis plant material using liquid CO₂. According to DE 103 37 458 A1, the use of liquid CO₂ is superior to super-critical CO₂, as it allows improved separation of waxes from the overall plant material mixture. However, using liquid CO₂ or super-critical CO₂ special equipment is required to generate and control the high pressures need to work with liquid or supercritical CO₂. Furthermore, in order to remove the aforementioned waxes the process described in DE 103 37 458 A1 requires post-processing of the obtained extract by subjecting the extract to treatment with ethanol at −20° C. for about 48 hours at a ratio of 2:1 (ethanol to extract).

The post-processing step of DE 103 37 458 A1, employing ethanol (or any other alcoholic solvent at low temperatures), is commonly named as “winterization”, (which is also required in processes using supercritical CO₂, as described e.g. in WO 2002/064109 A2 or any other extraction process using organic and/or alcoholic extraction of Cannabis plant material, e.g. WO 2013/165251).

Thus, all extraction processes, that use the principle of having the cannabinoids in the extract, regardless of solvent used, require subsequent removal of said waxes from the extract by means of winterization or analogous post-processing techniques. Alternative extraction technologies, inverting the principle and extracting the water-soluble substances from the plant material to result in an (inevitably solid) residue being enriched in cannabinoids are known as well. They have been subject to improvements and related publications however have a different objective than trying to obtain a product that can readily be used in a regulated, pharmaceutical context. For example, a process has been described that uses cut Cannabis plants or cut Cannabis flowers that have been cleared from stems and leaves of the plants and then subjected to cold water in semi-permeable bags (Cervantes, J., “Marijuana Horticulture, the indoor/outdoor medical grower's bible”, Van Patten Publishing 2006, p. 402; “Ice-O-Lator instructions”). These techniques, however, do not remove waxes from the fraction containing the cannabinoids.

Accordingly, removing the aforementioned waxes from the relevant oily phase has thereby become the actual problem in the generation of Cannabis plant extracts.

From the foregoing, it is apparent that there is still a need for an improved process for the production of an extract from Cannabis plants suitable for subsequent pharmaceutical use. Such processes should also maximize the content of the pharmaceutically active cannabinoids (or their precursors) and should maintain the concentration relative to each other. In other words, the extracts are preferably substantially free of waxes and other non-specific lipid soluble material but preferably contain substantially all of the cannabinoids naturally present in the plant, most preferably in substantially the same ratios in which they occur in the intact Cannabis plant. In the context of the present invention the extracts, preferably the Cannabis plant extract or Cannabis plant soft extract as obtainable according to the herein described methods comprise(s) preferably less than 3%, 2.1% or 2% waxes by weight.

Furthermore, the process should involve minimum use of solvents and energy for environmental, commercial as well as pharmaceutical reasons. Thus, there is a need for improved means and methods for the production of Cannabis plant extracts.

### SUMMARY OF INVENTION

The technical problem underlying the present invention is the provision of improved means and methods for the production of Cannabis plant extracts.

The technical problem is solved and the above-mentioned needs are addressed by the provisions of the embodiments characterized in the claims and as provided herein below.

The invention relates to a method for producing a Cannabis plant extract comprising delta-9-tetrahydrocannabinol (THC) from a Cannabis plant comprising the following steps:
(a) providing a Cannabis plant which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract comprising delta-9-tetrahydrocannabinol (THC) from the flower material and the solvent.

Furthermore, the invention relates to a Cannabis plant extract comprising delta-9-tetrahydrocannabinol (THC) as obtainable by/produced by the methods described herein. The Cannabis plant extract as obtainable by the herein described methods may be also referred to as Cannabis plant extract (hereinafter also termed “Cannabis plant soft extract”).

The method according to the invention is characterized by the fact that the plant material used is treated in such a way, so that the material that enters the subsequent alcoholic and/or organic extraction step, consists mainly of the blossoms/flower material, or preferably only of the blossoms/flower material. In the context of the present invention the term “consists mainly” is understood in such a way that up to 1%, 2%, 3%, 4%, 5%, 6%, 7%, 8%, 9%, 10%, 11%, 12%, 13%, 14%, 15%, 16%, 17%, 18%, 19% or 20% by weight of the plant material is plant material such as leaves, roots, etc. In other words, the term “consists mainly” means that up to 80%, 81%, 82%, 83%, 84%, 85%, 86%, 87%, 88%, 89%, 90%, 91%, 92%, 93%, 94%, 95%, 96%, 97%, 98%, 99%, or 100% by weight of the plant material is flower material.

The present invention solves the above identified technical problem since as documented herein a method has been surprisingly and unexpectedly found that obviates the need for a winterization step. Accordingly, the invention relates to a method for producing a Cannabis plant extract without a winterization step. In other words, the invention relates to method for producing a Cannabis plant extract that does not comprise a winterization step. The absence of a winterization step reduces the amount of (organic) solvents required for the production of the Cannabis plant extract. Furthermore, the absence of a winterization step reduces the amount of energy required for the production of the Cannabis plant extract. Despite the absence of the winterization step the Cannabis plant extract or Cannabis plant soft extract produced by/obtainable by the methods described herein contains excellent concentrations of pharmaceutically active substances such as cannabinoids. Despite the absence of the winterization step the Cannabis plant extract produced by/obtainable by the methods described herein contains excellent concentrations of THC (such as at least about 70 percent by weight of the Cannabis plant extract). Furthermore, the Cannabis plant extract produced by the methods described herein contains excellent and highly desired concentrations of pharmaceutically active substances relative to each other. In addition, the Cannabis plant extract produced by the methods described herein does not contain or contains only very low amounts of saponifiable substances (e.g. less than about 8% by weight of the extract). Accordingly, the Cannabis plant extract produced by the methods described herein is a readily, pharmaceutically usable extract.

This is particularly surprising, since the methods described herein use blossoms/flower material of Cannabis, which usually comprise the largest amounts of cannabinoids but also waxes. Accordingly, it was completely unexpected and surprising that the winterization step can be omitted without affecting quality of the obtained Cannabis plant extract. Furthermore, the methods described herein allow to produce Cannabis plant extracts with excellent concentrations of THC (such as at least about 70 percent by weight of the Cannabis plant extract) using ethanol as a solvent. The use of ethanol allows that simpler equipment is used compared to e.g. supercritical CO₂ as used in methods of the prior art.

The methods described herein allow to produce Cannabis plant extracts with desired concentrations of pharmaceutically active substances. The methods described herein allow to produce Cannabis plant extracts with desired concentrations of cannabinoids. Thus, the methods described herein allow to produce Cannabis plant extracts with desired concentrations of CBD and/or THC (A), preferably delta-9-tetrahydrocannabinol. The methods described herein allow to produce Cannabis plant extracts with desired concentrations of cannabinoids (preferably delta-9-tetrahydrocannabinol) and/or terpenes. The methods described herein allow advantageously to produce Cannabis plant extracts with desired concentrations of pharmaceutically active substances as mentioned herein and directly above without or with very low amounts of undesired waxes. Surprisingly and unexpectedly the methods described herein allow to produce said Cannabis plant extracts without a winterization step.

In the following the invention is described in more detail.

In particular the invention relates to the following items.

[1] A method for producing a Cannabis plant extract comprising delta-9-tetrahydrocannabinol (THC) from a Cannabis plant comprising the following steps:
(a) providing a Cannabis plant which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract comprising delta-9-tetrahydrocannabinol (THC) from the flower material.

[2] The method of item 1, wherein the method further comprises evaporating the solvent from the liquid Cannabis plant extract to concentrate the extract.

[3] The method of item 2, wherein the method further comprises heating the concentrated Cannabis plant extract to a temperature of 50° C. to 150° C., preferably 80° C. to decarboxylate tetrahydrocannabinolic acid (THCA) to delta-9-tetrahydrocannabinol (THC), preferably under vacuum at a pressure of 175 mbar to 195 mbar, more preferably under vacuum at about 185 mbar.

[4] The method of any one of items 1 to 3, wherein the Cannabis plant comprises delta-9-tetrahydrocannabinol (THC) in an amount of at least 1, 2, 3, 4, 5, 6, 7, 8, 9, or preferably 10 percent by dry weight (w/w).

[5] The method of any one of items 1 to 4, wherein the drying step of the flower material of the Cannabis plant as defined in step (b) of item 1 is performed at a temperature range of 20° C. to 35° C. for at least 4 days (96 h) or until the water content of the flower material is below 10 percent.

[6] The method of any one of items 1 to 5, wherein the treating step as defined in step (c) of item 1 comprises the following steps:
(i) overlaying the flower material with a solvent in a ratio of about 1:8.4 (w/w) thereby obtaining a mixture of the flower material and the solvent;
(ii) performing maceration for about 24 h at a temperature of 15° C. to 25° C. and exclusion of light;
(iii) adding further solvent thereby obtaining a mixture of the flower material and solvent in a ratio of about 1:16.8 (w/w);
(iv) performing percolation, wherein the solvent is seeped through the flower material with a flow rate of 3.55 to 5.45 ml/min per kg flower material, preferably 4.0 ml/min per kg flower material for 63 to 97 h, preferably for 86 h at a temperature of 15° C. to 25° C. and exclusion of light; and
(v) separating the Cannabis plant extract from the Cannabis flower material.

[7] The method of any one of items 1 to 6, wherein the separating step as defined in step (v) of item 6 comprises filtering the Cannabis plant extract with a deposition rate of 1.5 μm.

[8] The method of any one of items 1 to 7, wherein the solvent is selected from the group consisting of ethanol, butanol, alkanes (such as pentane, heptane and propane), ethyl ether, tert butyl-methyl-ether, methyl-ethyl-ketone, acetone, ethyl acetate and CO₂, preferably ethanol, more preferably ethanol 96 vol. % of pharmaceutical grade.

[9] The method of any one of items 1 to 8, wherein the Cannabis plant is DKJ127 (deposited by the Community Plant Variety Office with the application number A202104053).

The method of any one of items 1 to 9, wherein the method does not comprise a winterization step.

A Cannabis plant extract comprising delta-9-tetrahydrocannabinol (THC) as obtainable by the method of any one of items 1 to 10.

The Cannabis plant extract of item 11, wherein the plant extract is in liquid form.

The Cannabis plant extract of item 11 or 12, wherein the plant extract is in solvent-free and decarboxylated form.

The Cannabis plant extract of any one of items 11 to 13, or as obtainable by the method of any one of items 2 to 10, wherein the Cannabis plant extract comprises delta-9-tetrahydrocannabinol (THC) in an amount of at least about 50, 60 or 70 percent by weight of the extract, preferably between about 60 and 80 percent by weight of the extract, more preferably between about 70 and 74 percent by weight of the extract.

The Cannabis plant extract of any one of items 11 to 14, or as obtainable by the method of any one of items 1 to 10, wherein the Cannabis plant extract further comprises one or more terpene(s) selected from the group consisting of alpha-bisabolol, guaiol, and beta-caryophyllene.

The Cannabis plant extract of any one of items 11 to 15, or as obtainable by the method of any one of items 1 to 10 for use in medicine.

The Cannabis plant extract of any one of items 11 to 15, or as obtainable by the method of any one of items 1 to 10 for use in the treatment and/or prevention of chronic cancer pain, somatic pain, visceral pain, central neuropathic pain, peripheral neuropathic pain or complex pain syndromes.

A method treatment and/or prevention of chronic cancer pain, somatic pain, visceral pain, central neuropathic pain, peripheral neuropathic pain or complex pain syndromes comprising administering the Cannabis plant extract of any one of items 11 to 15, or as obtainable by the method of any one of items 1 to 10.

The disclosures in the context of the methods of the invention described herein are applicable to the corresponding uses and vice versa.

### BRIEF DESCRIPTION OF THE FIGURES

FIG. 1: The Cannabis plant soft extract as obtained by the herein described method was analysed as described in section 6 of the Example section. D9-THC=delta-9-THC

### DETAILED DESCRIPTION

As mentioned above, the invention relates to a method for producing plant extracts. This means that an extract is produced from/obtained from plant material. In other words, a plant extract is produced by/obtained by extracting plant material according to the methods described herein. In particular, the invention relates to a method for producing (a) Cannabis plant extract(s). This means that an extract is produced from Cannabis plants. In other words, a Cannabis plant extract is produced by extracting Cannabis plant material according to the methods described herein.

Accordingly, the invention relates to a method for producing a Cannabis plant extract comprising the following steps:
(a) providing a Cannabis plant, preferably which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract from the flower material.

The invention also relates to a method for producing a Cannabis plant extract comprising the following steps:
(a) providing a Cannabis plant, preferably which comprises delta-9-tetrahydrocannabinol (THC) in a sufficient amount;
(b) trimming and drying the flower material separated from the remaining plant material to obtain a trimmed and dry plant material; and
(c) treating the flower material of step (b) with a solvent and separating the Cannabis plant extract from the flower material.

The invention further relates to a method for producing a Cannabis plant extract comprising the following steps:
(a) providing a Cannabis plant, preferably which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract from the mixture of the flower material and the solvent;
wherein the treating step as defined in step (c) comprises the following steps:
(i) overlaying the flower material with a solvent in a ratio of about 1:8.4 (w/w) thereby obtaining a mixture of the flower material and the solvent;
(ii) performing maceration for about 24 h at a temperature of 15° C. to 25° C. and exclusion of light;
(iii) adding further solvent thereby obtaining a mixture of the flower material and solvent in a ratio of about 1:16.8 (w/w);
(iv) performing percolation, wherein the solvent is seeped through the flower material with a flow rate of 3.55 to 5.45 ml/min per kg flower material, preferably 4.0 ml/min per kg flower material for 63 to 97 h, preferably for 86 h at a temperature of 15° C. to 25° C. and exclusion of light; and
(v) separating the Cannabis plant extract from the Cannabis flower material.

In context of the present invention it is envisaged that the steps (i) to (v) are performed in subsequent order.

In the context of the present invention, the term “plant” refers to any various photosynthetic, eukaryotic multicellular organisms of the kingdom Plantae, characteristically producing embryos, containing chloroplasts, having cellulose cell walls and lacking locomotion. As used herein, a “plant” includes any plant or part of a plant at any stage of development and progeny thereof containing cannabinoid, terpene and terpenoid compounds. The plant material may be obtained from solid plant material but is not limited thereto. The solid plant material may be obtained from the whole plant or parts thereof, which includes, without limitation, plant cells, plant organs, leaves, stems, fruits, roots, meristems, plant seeds, protoplasts, callus, and any groups of plant cells organized into structural and/or functional units. Alternatively, the plant material may be a plant cell culture, specifically a plant suspension cell culture in a liquid medium. Methods to establish an in vitro plant cell culture from solid plant material are known to those skilled in the art and include callogenesis (Espinosa-Leal et al., Planta (2018), 248, pp. 1-18). It is evident for the skilled person that what is described herein for plants in general may also apply to Cannabis plants.

“Cannabis plant” may be used interchangeably with “Cannabis plant material”. Any part of the Cannabis plant may be used, including but not limited to trichomes, flower buds, flower bracts, leaves, stalk and any other plant part that may contain cannabinoids. Preferred Cannabis plant parts that contain cannabinoids are flowers, trichomes and sugar leaves. Also, although the female plants may produce a higher concentration of cannabinoids than male plants, both female (including “feminized plants”) and male plants can be used. The terms “flower”, “flower material” and “blossom” may be used interchangeably herein. The skilled person knows that in Cannabis plants the flowers are arranged in so called colas. Accordingly, flower material as used herein also refers to said colas.

The leaves of the Cannabis plants may be distinguished in fan leaves and sugar leaves. Fan leaves are the large, primary leaves on the Cannabis plant. Sugar leaves develop and grow out of Cannabis flowers in the plant's flowering stage. As described in detail below said sugar leaves or parts thereof may be removed from the flower material in the herein described methods.

Trichomes (i.e. resin glands) of the Cannabis plant material are nearly microscopic, mushroom-like protrusions from the surface of the Cannabis plant, mainly of the flower buds. While relatively complex, trichomes are comprised primarily of a stalk and a head. The production of cannabinoids such as THC occurs predominantly in the head of the trichome. Cannabinoids are concentrated in the trichomes of the plant. Accordingly, it is evident for the skilled person that all herein described methods may be performed using (isolated) trichomes of Cannabis plants. Isolation of trichomes may be performed as described in the prior art (e.g. Cerantes, J., “Marijuana Horticulture, the indoor/outdoor medical grower's bible”, Van Patten Publishing 20″6, p. 402; “Ice-O-Lator Instructions”).

In the present invention, when a Cannabis plant is used for the herein described methods, the plant material is preferably obtained from the flowers of the plant. Further, in the context of the present invention, the plant material used for the herein described methods may be obtainable from a native or a transgenic plant.

In the context of the present invention, the plant material used to obtain the extract may be fresh, dried, freeze dried or frozen, but is preferably dried.

“Drying” as used herein means that the water/moisture in the plant material is reduced. Drying of the plant material can be performed by any means for example in a drying chamber by placing the plant material on trays through which air flows at a temperature range of 20° C. to 35° C. (i.e. 20° C., 21° C., 22° C. 23° C., 24° C., 25° C., 26° C., 27° C., 28° C., 29° C., 30° C., 31° C., 32° C., 33° C., 34° C., 35° C. or any fraction thereof) for at least 1 day, 2 days, 3 days or 4 days, preferably 4 days (96 h) or until the water content of the flower material is below 10 percent. The skilled person is aware how to measure the temperature via standard methods. Furthermore, the skilled person knows how to determine the water content in plant material, in particular in dried Cannabis flowers (e.g. as described in European Pharmacopeia 10th edition, chapter 2.2.32). Accordingly, the drying step of the flower material of the Cannabis plant as defined in step (b) of the herein described methods may be performed for example in a drying chamber by placing the plant material, preferably flower material on trays through which air flows at a temperature range of 20° C. to 35° C. (i.e. 20° C., 21° C., 22° C., 23° C., 24° C., 25° C., 26° C., 27° C., 28° C., 29° C., 30° C., 31° C., 32° C., 33° C., 34° C., 35° C. or any fraction thereof) for at least 1 day, 2 days, 3 days or 4 days, preferably 4 days (96 h) or until the water content of the flower material is below 10 percent.

Preferably, the plant material is trimmed. Accordingly, the flower material used in the herein described methods is trimmed.

The term “trimming of the flower material” as used herein means that the leaves, particularly the sugar leaves and/or fan leaves, are removed from the flowers. In particular the visible parts of the sugar leaves that protrude from the flower are cut off. Trimming of the flower material may be performed as described in the following.

The flowers of the Cannabis plant may be separated from the rest of the plant manually (e.g. with a scissors) or with a destalker (e.g. MB Bucker 500; Master Bucker 500; www.masterproducts.es).

Then the undried flowers are trimmed to separate the sugar leaves from the flower. This step may be done manually (e.g. with a scissors) or with a trimmer (e.g. MT Tumbler 500 MED; MT Tumbler 500 MED; masterproducts.es).

A trimmer that may be used in the herein described methods may function as follows:
(a) The undried or dried flowers are placed in a rotating drum made of stainless steel.
(b) The speed of the transport of the flowers through the drum can be adjusted with the help of the angle of inclination of the drum. The flowers are also transported towards the exit by the newly added flowers. About a handful of flowers are added every 30 seconds and remain in the drum for about 30 to 45 seconds.
(c) Sugar leaves that protrude through the openings of the drum are removed by a roller blade which is attached under the drum.

In order to increase the throughput per minute, e.g. several trimmers can be connected in series and material can be added at shorter intervals.

Removal of the sugar leaves is optional. In contrast to the fan leaves, there are also smaller amounts of trichomes on the surface of the sugar leaves. The sugar leaves may be removed to achieve the highest possible THC content in the raw material for extraction, as the percolator (extraction container) can only be loaded with a defined amount of material. The skilled person can readily choose suitable amount of plant material to be loaded into the percolator. For example, for a 1,000 liters percolator 10 to 25 kg plant material may be used.

In addition to trimming the plant material used for the herein described methods may be broken down to produce a plant material of a smaller size. In other words, the plant material may also be mechanically comminuted during mixing with the solvent or preferably prior to mixing with the solvent. All comminution methods known to the person skilled in the art are suitable that reduce the plant material into smaller pieces including smashing, pulverizing, milling, grinding, and chipping. However, in the herein described methods it is preferred that whole flowers from which the visible parts of the sugar leaves have been trimmed are used. Cannabinoids and terpenes are located on the surface of the flower and easily accessible for extraction solvents without comminution. Without necessarily being bound by scientific theory it is envisaged that smaller particles increase the contact surface with solvent that may lead to a higher extraction of undesired substances/material. In other words, extraction of smaller parts may result in the extraction of a higher level of non-active plant components.

As mentioned above it is preferred that a Cannabis plant extract is produced by/obtainable by the methods described herein. Accordingly, a Cannabis plant extract is produced by extracting Cannabis plant material according to the methods described herein.

The term “Cannabis plant(s)” is used herein in the broadest sense and includes the wild-type Cannabis sativa and Cannabis indica and all variants thereof, such as Cannabis sativa subspecies indica including the variants var. indica and var. kafirinistanica, Cannabis indica subsp. indica var. indica, Cannabis indica subsp. indica var. himalayensis, Cannabis indica subsp. indica var. afghanica and Cannabis indica subsp. indica var. asperrima. Furthermore, it includes the Cannabis plants resulting from genetic crosses, self-crosses or hybrids of the above-mentioned plants and Cannabis chemovars. From said Cannabis plants all kind of the above-mentioned plant material can be used for the methods described herein. Preferably, the flower material is used for the methods described herein. The terms “Cannabis variety” and “Cannabis plant variety” are used interchangeably herein. Cannabis varieties are known in the art, such as Jack Herer, Chemdawg, Bubba Kush, Trainwreck, Super Silver Haze, Pure Kush, El Nino, Himalayan Gold, Skunk #1, White Widow, Warlock CBD, Pink Kush, OG Kush, Super Lemon Haze, Jack the Ripper, Lemon Skunk, and Hash Plant.

The preparation of convenient ratios of THC- and CBD-containing medicines, i.e. Cannabis plant extracts is made possible by the cultivation of specific chemovars of Cannabis. These chemovars (plants distinguished by the cannabinoids produced, rather than the morphological-characteristics of the plant) can be bred by a variety of plant breeding techniques which will be familiar to a person skilled in the art. Propagation of the plants by cuttings for production material ensures that the genotype is fixed and that each crop of plants contains the cannabinoids in substantially the same ratio.

Cannabis plants naturally produce a diverse array of secondary metabolites, including cannabinoids, terpenes and terpenoids, sterols, triglycerides, alkanes, squalenes, tocopherols, and others. The mix of these secondary metabolites varies depending on several factors, including the specific Cannabis variety, the parts of the Cannabis plant to be extracted, the method of extraction and the processing of the extract.

Accordingly, the skilled person is well aware that different Cannabis varieties contain different amounts/concentrations of (pharmaceutically active) substances such as cannabinoids or terpenes. Accordingly, it is evident for the skilled person that the concentration of (pharmaceutically active) substances such as cannabinoids or terpenes in the Cannabis plant extract produced by the methods described herein can be controlled by using Cannabis plant varieties with a certain concentration of (pharmaceutically active) substances. For example, a Cannabis plant extract comprising a high concentration of THC may be produced by using a Cannabis plant variety comprising a high concentration of THC whereas a Cannabis plant extract comprising a high concentration of CBD may be produced by using a Cannabis plant variety comprising a high concentration of CBD. Accordingly, the skilled person may choose the Cannabis plant variety according to the ratio of e.g. cannabinoids relative to each other. The precise cannabinoid content of any particular Cannabis variety may be qualitatively and quantitatively determined using methods well known to those skilled in the art, such as Thin Layer Chromatography (TLC) or High Performance Liquid Chromatography (HPLC).

Any Cannabis variety mentioned herein or known in the art may be used for the herein described methods. Although evident for the skilled person it is noted that also a mix of different Cannabis varieties may be used for the methods described herein.

The Cannabis plant used in the herein described methods may be obtained via selection on high THC concentrations. Exemplarily, a selection could be done as follows: Cannabis plant seeds, e.g. of the strain “Jack Herer”, may be commercially obtained. The seeds may be cultivated by ordinary cultivation methods known to the skilled person, e.g. for between 3 and 14 days until germination. Based on germination and growing behavior, the skilled person can select specific seeds for further cultivation. Such decisions may generally be made based upon analytical results and/or observations by the cultivar during cultivation. Once the selected seeds are germinated and rooted, they may be further cultivated for three to six weeks by ordinary cultivation methods known to the skilled person to establish a stock of seed plants. Fertilization, water supply, pest control, disease monitoring and trimming of the plants may be optimized by a skilled person. Based on growing behaviour, the skilled person can select specific seed plants for further cultivation. From these selected plants (in the following passage referred to as individual plants), cuttings may be taken to secure genetic material of the individual plant. The individual plants may then be brought into flowering phase by reducing daylength from >18 hours to <12 hours of daylight. Generally, the environmental conditions for rooting, vegetative and flowering phase need to be fulfilled. Then, the individual plants may finally be selected based on the following criteria: growing behavior, flowering behavior, successful cultivation and good rooting behavior of cuttings taken therefrom, susceptibility to diseases (e.g. grey mould) and analytical results for e.g. THC content, content of other cannabinoids and terpene profile. As part of the selection process, batch-to-batch consistency may be analysed based on cannabinoid and terpene profile. Most preferably, the Cannabis variety used for the methods described herein is the variety referred to in here as DKJ127 and deposited by the Community Plant Variety Office (deposited by Vertanical GmbH with the application number A202104053 (provisional designation of the variety: dk-j127; Botanical taxon: Cannabis sativa L.; Breeder's reference: DK-J127; Variety denomination: DKJ127; Application No.: 2021/3223; Electronic Application No: A202104053; Date of receipt by the Community Plant Variety Office: Sep. 12, 2021)). Although evident for the skilled person it is noted that also a progeny of the variety DKJ127 may be used for the herein described methods. Said progeny may be selected for even higher THC concentrations than the deposited DKJ127.

Accordingly, the invention relates to a method for producing a Cannabis plant extract comprising the following steps:
(a) providing a Cannabis plant of the Cannabis variety DKJ127 (i.e. as deposited by the Community Plant Variety Office with the application number A202104053);
(b) trimming and drying the flower material separated from the remaining plant material; and
(c) treating the flower material of step (b) with a solvent and separating the Cannabis plant extract from flower material.

The invention further relates to a method for producing a Cannabis plant extract comprising the following steps:
(a) providing a Cannabis plant of the Cannabis variety DKJ127;
(b) trimming and drying the flower material separated from the remaining plant material; and
(c) treating the flower material of step (b) with a solvent and extracting the Cannabis plant extract;
wherein the treating step as defined in step (c) comprises the following steps:
(i) overlaying the flower material with a solvent in a ratio of about 1:8.4 (w/w) thereby obtaining a mixture of the flower material and the solvent;
(ii) performing maceration for about 24 h at a temperature of 15° C. to 25° C. and exclusion of light;
(iii) adding further solvent thereby obtaining a mixture of the flower material and solvent in a ratio of about 1:16.8 (w/w);
(iv) performing percolation, wherein the solvent is seeped through the flower material with a flow rate of 3.55 to 5.45 ml/min per kg flower material, preferably 4.0 ml/min per kg flower material for 63 to 97 h, preferably for 86 h at a temperature of 15° C. to 25° C. and exclusion of light; and
(v) separating the Cannabis plant extract from the Cannabis flower material.

Cannabinoids are described to be unique terpenophenolic metabolites found only in Cannabis plants (Sirikantaramas and Taura, Springer 2017, 1st edition, Chapter 8, p. 183-206). However, alternative cannabinoid-like substances or cannabimimetic compounds can be found in other plant species including Acmella oleracea (Dallazan et al., Inflammopharmacology 2019, 28, pp. 175-186), Echinaceae angustifolia (Raduner et al., J Biol Chem. 2006, 281 (20), pp. 14192-14206), Echinaceae purpurea (Raduner et al., J Biol Chem. 2006, 281 (20), pp. 14192-14206), Helichrysum umbraculigerum (Pollastro et al., Fitoterapia 2018, 126, pp. 35-39), Heliopsis helianthoides (Hajdu et al., J Nat Prod. 2014, 77(7), pp. 1663-1669), Lepidium meyenii (Hajdu et al., J Nat Prod. 2014, 77(7), pp. 1663-1669), Piper methysticum (Ligresti et al., Pharmacol Res. 2012, 6(2), pp. 163-169), Piper nigrum (Reynoso-Moreno et al., J Agric Food Chem. 2017, 65(43), pp. 9435-9442), Radula marginata (Hussain et al., Phytochem rev. (2019), 18, pp. 953-965), Radula perrottetii (Chicca et al., Neurophysiol. 2018, 4(10)), Rhododendron anthopogonoides (Iwata and Kitanaka, Chem Pharm Bull. 2011, 59(11), pp. 1409-1412) and Tuber melanosporum (Degenhardt et al., Biology, Pharmacology, Diagnosis, and Treatment 2017, Chapter 2, pp. 13-23), (Pacioni et al., Phytochemistry 2015, 110, pp.: 104-110). Cannabinoid-like substances or cannabimimetic compounds are considered as phytochemicals and secondary metabolites able to interact with the endocannabinoid system and having similar pharmacological effects to cannabinoids.

In the context of the present invention, the term “extract”, without further specification, is intended to generally refer to any form of the product of extraction, optionally minus the extracting agent, regardless of the physical form (i.e. viscous, pasty or solid).

Alternatively, the methods described herein may use a plant that is capable of producing cannabinoids, cannabinoid-like substances or cannabimimetic compounds as well as terpenes and/or terpenoids, but does not endogenously contain or produce them. That means that the plant used for the methods described herein may be a transgenic plant that has been genetically modified to produce the desired substance (e.g. cannabinoids and/or terpenes and/or terpenoids). As such, the skilled person will understand that the plants used in the herein described methods may be transgenic plants or plant cells which differ from naturally occurring ones due to genetic modification. Genetically modified plants or plant cells do not naturally occur, i.e., cannot be found in nature, and differ substantially from naturally occurring plants or plant cells due to the introduction of foreign genetic material, for example a foreign nucleic acid molecule.

It is envisaged herein that the herein described methods are for producing a plant extract, preferably a Cannabis plant extract comprising (pharmaceutically active) substances e.g. cannabinoids, terpenes and/or terpenoids. The term “cannabinoid” as used herein relates to any cannabinoid that has been isolated from a plant or has been synthetically created to have activity in the endocannabinoid system and includes cannabinoid-like substances and/or cannabimimetic compounds. Cannabinoids synthesized by plants sources are considered to be phytocannabinoids, i.e. plant-based cannabinoids. In the context of the present invention, the term “cannabinoid” may be interchangeably used with “phytocannabinoids”. The term “cannabinoid profile” is used to describe the combination of cannabinoid, cannabinoid-like substances or cannabimimetic compounds present in the plant extract. To date, over 100 cannabinoids have been identified in Cannabis plants. A comprehensive, non-limiting list of such cannabinoids in Cannabis may be found in ElSohly M. A. and Gul W., in Handbook of Cannabis, Oxford University Press (2014), pp. 3-22. A preferred but non-limiting example for cannabinoids is delta-9-tetrahydrocannabinol (THC).

References to “THC” or “delta-9-tetrahydrocannabinol” and “Cannabidiol” or “CBD” or “cannabinoid(s)” as used herein, will be understood to also encompass pharmaceutically acceptable salts of such compounds. The term “pharmaceutically acceptable salts” refers to salts or esters prepared from pharmaceutically acceptable non-toxic bases or acids, including inorganic bases or acids and organic bases or acids, as would be well known to persons skilled in the art. Many suitable inorganic and organic bases are known in the art.

The term “terpene(s)” or “terpenoid(s)” as used herein refers to a class of hydrocarbon molecules (Radwan, M. M. et al. Molecules 2021, 26(9): 2774). Non-limiting examples for terpenes are alpha-bisabolol, guaiol and beta-caryophyllene. Further non-limiting examples of terpenes are provided herein below. Terpenoids are terpene compounds that have been further metabolized in the plant, typically through an oxidative process, and therefore usually contain at least one oxygen atom (Radwan, M. M. et al. Molecules 2021, 26(9): 2774). The term “terpene profile” is used to describe the combination of terpene and terpenoid compounds present in the plant extract.

It is envisaged herein that the methods described herein are for producing plant extracts comprising e.g. cannabinoids, terpenes and/or terpenoids by using any plant material, which is known to contain said substances. As mentioned before a preferred plant material are Cannabis plants, preferably flower material of Cannabis plants.

The methods described herein are preferably for producing Cannabis plant extracts comprising delta-9-tetrahydrocannabinol (THC).

Accordingly, the invention relates to a method for producing a Cannabis plant extract comprising delta-9-tetrahydrocannabinol (THC) from a Cannabis plant comprising the following steps:
(a) providing a Cannabis plant which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract comprising delta-9-tetrahydrocannabinol (THC) from the flower material.

The invention relates to a method for producing a Cannabis plant extract comprising delta-9-tetrahydrocannabinol (THC) from a Cannabis plant comprising the following steps:
(a) providing a Cannabis plant which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract comprising delta-9-tetrahydrocannabinol (THC) from the flower material;
wherein the drying step of the flower material of the Cannabis plant as defined in step (b) is performed in a drying chamber by placing the plant material on trays through which air flows at a temperature range of 20° C. to 35° C. for at least 1 day, 2 days, 3 days or 4 days, preferably 4 days.

The invention also relates to a method for producing a Cannabis plant extract comprising delta-9-tetrahydrocannabinol (THC) from a Cannabis plant comprising the following steps:
(a) providing a Cannabis plant which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract comprising delta-9-tetrahydrocannabinol (THC) from the flower material;
wherein the drying step of the flower material of the Cannabis plant as defined in step (b) is performed in a drying chamber by placing the plant material on trays through which air flows at a temperature range of 20° C. to 35° C. until the water content of the flower material is below 10 percent.

Methods for determining the water content may be found in European Pharmacopeia (2.2.32) and the German monograph on Cannabis flos. For example, it may be determined with 1.0 g of powdered drug by drying for 24 hours in vacuo over molecular sieve R at 40° C. and a pressure between 1.5 and 2.5 kPa.

The term “sufficient amount” as used herein means that the plant, preferably the Cannabis plant has an amount of THC sufficiently high that the plant extract, preferably the Cannabis plant extract produced by the herein described methods using said plant or Cannabis plant has the desired amount of THC. In other words, “sufficient amount” as used herein means that the plant, preferably the Cannabis plant has a concentration of THC sufficiently high that the plant extract, preferably the Cannabis plant extract or Cannabis plant soft extract produced by the herein described methods using said plant or Cannabis plant has the desired concentration of THC. In general, “sufficient amount” as used herein means that the Cannabis plant has a concentration of THC of at least 1, 2, 3, 4, 5, 6, 7, 9, 10, 20 or 30 percent by dry weight (w/w). Dry weight as used herein means that the plant material is water free. For example: If the THC content is measured for a sample with a water content of 10% and the measured THC concentration for the sample is 20% THC the THC concentration by dry weight would be 22.2%.

The skilled person is well aware how the THC content of Cannabis plant or flower can be determined (e.g. German Pharmacopeia 2018—Cannabisblüten/Cannabis flos, Announcement in the Federal Gazette: BAnz AT 24.04.2018 B5).

In general, when a Cannabis plant with a concentration of THC of at least 1, 2, 3, 4, 5, 6, 7, 8, 9, 10, 20, or 30 percent by dry weight (w/w) is used in the herein described methods the obtained/produced Cannabis plant extract (or the Cannabis plant soft extract e.g. after concentration) may have a THC concentration of at least 60, preferably about 70 to 74 percent by weight of the Cannabis plant extract. As already mentioned herein the skilled person is well aware that different Cannabis varieties contain different amounts/concentrations of cannabinoids (preferably THC) and/or other (pharmaceutically active) substances (e.g. terpenes). Accordingly, the skilled person may choose the Cannabis varieties used in the herein described methods depending on the desired concentrations of cannabinoids (preferably THC) and/or other (pharmaceutically active) substances (e.g. terpenes) in the Cannabis plant extract produced by said methods. Of course, the skilled person is also well aware that different part of plants as described elsewhere herein have different concentrations of e.g. cannabinoids (preferably THC) and/or other (pharmaceutically active) substances (e.g. terpenes). For example, in Cannabis plants the concentration of THC and/or other (pharmaceutically active) substances (e.g. terpenes) is usually high in the flower material.

Accordingly, it is evident for the skilled person that when e.g. only flower material is used in the herein described methods to produce a Cannabis plant extract the concentration of THC in the flower material may be relevant and the skilled person may chose the used Cannabis variety according the concentration of THC in the flower material and not according to the corresponding concentrations in the whole plant.

The concentration/amount of e.g. cannabinoids (e.g. THC and/or CBD) and/or other (pharmaceutically active) substances (e.g. terpenes) in the plant, preferably Cannabis plant used in the herein described methods may be determined as percent of e.g. cannabinoids (e.g. THC and/or CBD) and/or other (pharmaceutically active) substances (e.g. terpenes) by fresh weight or dry weight, preferably dry weight. Accordingly, the concentration/amount of THC in the Cannabis plant may be determined as percent THC by dry weight. The concentration/amount of THC in the Cannabis plant may be determined as percent THC by dry weight in the whole plant (i.e. leaf, flower and stem) or may be determined as percent THC by dry weight in the flower material only.

The concentration/amount of THC in the flower material of the Cannabis plant used in the herein described methods may be at least 1, 2, 3, 4, 5, 6, 7, 8, 9, 10, 20 or 30 percent by dry weight (w/w), preferably at least 10 percent by dry weight. It is evident for the skilled person that THC content as used herein may also refer to THC equivalents which is the sum of THC and THCA*0.877. Accordingly, in the context of the present invention, the term “THC content” encompasses the THC and THCA content. The THC and THCA content may be measured by liquid chromatography as described in the art (e.g. German Pharmacopeia (DAB) 2018, ISBN: 978-3-7692-7217-8; Danish Medicines Standards 2020.0, BEK Nr. 1231 af25/11/2019; https://www.retsinformation.dk/eli/Ita/2019/1231). Dry weight is defined as the weight of the material subtracted by the loss on drying (mainly water).

First the loss on drying is determined. The loss on drying is defined as the loss of mass after drying under specified conditions (for example in an oven) according to the European Pharmacopeia (2.2.32). The loss on drying is a measure of the water content (and other volatile compounds) which are removed during the drying step.

Preferably, the Cannabis plant used in the herein described methods comprises THC in an amount of at least 10 percent by dry weight (w/w).

Therefore, the invention relates to a method for producing a Cannabis plant extract comprising delta-9-tetrahydrocannabinol (THC) from a Cannabis plant comprising the following steps:
(a) providing a Cannabis plant which comprises delta-9-tetrahydrocannabinol (THC) in an amount of at least 10 percent by dry weight (w/w);
(b) trimming and drying the flower material separated from the remaining plant material; and
(c) treating the flower material of step (b) with a solvent and separating the Cannabis plant extract comprising delta-9-tetrahydrocannabinol (THC) from the flower material.

In the context of the present invention, the THC amount of any particular Cannabis plant as described herein may be qualitatively and quantitatively determined using methods well known to those skilled in the art, such as Thin Layer Chromatography (TLC) or High Performance Liquid Chromatography (HPLC).

In the herein described methods a Cannabis plant extract is produced by/obtainable by extraction. Any extraction method maybe used e.g. solvent extraction, distillation methods, pressing and sublimation, decoction, digestion, percolation, soxlethtation, maceration or any other appropriate extraction method known to the person skilled in the art and combinations thereof may be used.

Preferred herein is solvent extraction, i.e. extraction using a solvent. It is envisaged that the plant material, preferably the Cannabis plant material is brought into contact with the solvent, which may also be called the extractant. In other words, the plant material, preferably the Cannabis plant material is treated with a solvent.

It is envisaged that the desired (pharmaceutically active) substances from the plant material dissolve in the solvent. Accordingly, it is envisaged that the e.g. cannabinoids (preferably THC) and/or other (pharmaceutically active) substances (e.g. terpenes) from the Cannabis plant material dissolve in the solvent. That means that after the treatment of the Cannabis plant material with the solvent the e.g. cannabinoids (e.g. THC and/or CBD) and/or other (pharmaceutically active) substances (e.g. terpenes) are no longer present in the Cannabis plant material but are dissolved in the solvent. In other words, the solvent “removes” the e.g. cannabinoids (e.g. THC and/or CBD) and/or other (pharmaceutically active) substances (e.g. terpenes) from the plant material. In other words, the e.g. cannabinoids (preferably THC) and/or other (pharmaceutically active) substances (e.g. terpenes) are extracted from the plant material. The solvent with the dissolved e.g. cannabinoids (e.g. THC and/or CBD) and/or other (pharmaceutically active) substances (e.g. terpenes) can then be separated (i.e. extracted) from the plant material. The solvent with the dissolved e.g. cannabinoids (e.g. THC and/or CBD) and/or other (pharmaceutically active) substances (e.g. terpenes) separated from the plant material, preferably Cannabis plant material is called the plant extract, preferably Cannabis plant extract. The Cannabis plant extract may be further concentrated as disclosed elsewhere herein and may then be referred to as Cannabis plant soft extract. Preferably, the solvent used in the herein described methods removes THC from the plant material. Accordingly, the herein described methods may comprise the step of treating the (homogenized) flower material of step (b) with a solvent and separating the Cannabis plant extract comprising delta-9-tetrahydrocannabinol (THC) from the mixture of the flower material and the solvent. It is also preferred that the solvent used in the herein described methods removes THC and terpenes. Accordingly, it is preferred that the solvent used in the herein described methods removes THC and one or more of the terpenes selected from alpha-bisabolol, guaiol, and beta-caryophyllene.

As mentioned above in the herein described methods the Cannabis plant material may be treated with a solvent. Accordingly, the herein described methods may comprise overlaying the (homogenized) plant material with a solvent thereby obtaining a mixture of the plant material and the solvent. Said step is herein referred to as “mixture step”.

Likewise, the herein described methods may comprise overlaying the flower material of a Cannabis plant with a solvent thereby obtaining a mixture of the flower material of a Cannabis plant and the solvent. In particular, step (c) of the herein described methods may comprise overlaying the flower material of a Cannabis plant with a solvent thereby obtaining a mixture of the flower material of a Cannabis plant and the solvent. The skilled person can readily determine suitable ratios of plant material to solvent. A plant material to solvent ratio of 1:8.4 (w/w) as used herein means that 8.4 times the quantity of solvent relative to plant material according to weight is used. In other words, 1 g of plant material would be overlaid with 8.4 g solvent. The Cannabis plant material to solvent ratio used in the herein described methods may be as a non-limiting example all ratios between 1:0.1 (w/w) and 1:100 (w/w). Preferably, Cannabis plant material to solvent ratio used in the herein described methods is a ratio of 1:1 (w/w) to 1:20 (w/w) (i.e. 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 or 1:20 (w/w) or any fraction thereof, e.g. 1:14.7 (w/w)). Preferably, in the herein described methods the Cannabis plant material is overlaid with a solvent in a ratio of 1:8.4 (w/w) thereby obtaining a mixture of the Cannabis plant material and the solvent. Preferably, in the herein described methods the flower material of a Cannabis plant is overlaid with a solvent in a ratio of about 1:8.4 (w/w) thereby obtaining a mixture of the flower material and the solvent. Thus, it is preferred that step (c) of the herein described methods comprises overlaying the flower material with a solvent in a ratio of 1:8.4 (w/w) thereby obtaining a mixture of the flower material and the solvent.

It is preferred that the flower material is completely covered in solvent.

After a mixture of the plant material, preferably, Cannabis plant material, more preferably, flower material of a Cannabis plant, and the solvent is obtained the herein described methods may further comprise a step that allows the e.g. cannabinoids (preferably THC) and/or other (pharmaceutically active) substances (e.g. terpenes) to dissolve in the solvent. The mixture of the plant material, preferably, Cannabis plant material, more preferably, flower material of a Cannabis plant and the solvent may be incubated to allow the e.g. cannabinoids (preferably THC) and/or other (pharmaceutically active) substances (e.g. terpenes) to dissolve in the solvent. Said incubation step is preferably maceration (sometimes referred to as static extraction).

### Said Step is Herein Referred to as “Maceration Step”.

Maceration may be performed for minutes, hours, days or weeks, preferably hours to days. Accordingly, it may be performed for 1 h to 48 h (i.e. for 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 25 h, 26 h, 27 h, 28 h, 29 h, 30 h, 31 h, 32 h, 33 h, 34 h, 35 h, 36 h, 37 h, 38 h, 39 h, 40 h, 41 h, 42 h, 43 h, 44 h, 45 h, 46 h or 47 h, 48 h, preferably 24±1 hours (h)).

Said maceration may be performed at a temperature of 4° C. to 50° C., preferably, at 15° C. to 25° C. (i.e. at 15° C., 16° C., 17° C., 18° C., 19° C., 20° C., 21° C., 22° C., 23° C., 24° C., 25° C.).

Maceration is preferably performed under exclusion of light.

Accordingly, step (c) of the herein described methods may comprise performing maceration for about 24 h at a temperature of 15° C. to 25° C. The term “about” as used herein in connection with time means 10 percent more or 10 percent less than the denoted value.

Preferably, step (c) of the herein described methods may comprise performing maceration for 24±1 h at a temperature of 15° C. to 25° C. and exclusion of light.

It is further envisaged herein that further solvent is added after the mixture of the plant material, preferably, Cannabis plant material, more preferably, flower material of a Cannabis plant and the solvent was incubated e.g. via performing maceration. In other words, it is envisaged herein that further solvent is added after the “maceration step”.

Accordingly, step (c) of the herein described methods comprises adding further solvent to the mixture of the plant material, preferably, Cannabis plant material, more preferably, flower material of a Cannabis plant and the solvent. Said step is herein referred to as “further solvent step”. Identical or different solvent may be added. Preferably, an identical solvent is added to the mixture. Any amount of solvent may be added so that the Cannabis plant material to solvent ratio after further solvent has been added is e.g. 1:1 (w/w) to 1:20 (w/w) (i.e. 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 or 1:20 (w/w) or any fraction thereof, e.g. 1:14.7 (w/w)). Preferably, an amount of solvent is added so that the amount of solvent is doubled, i.e. when the Cannabis plant material to solvent ratio was 1:8.4 (w/w) before the further solvent is added the Cannabis plant material to solvent ratio after further solvent has been added is about 1:16.8 (w/w).

Preferably, after further solvent has been added the plant material, preferably, Cannabis plant material, more preferably, flower material of a Cannabis plant and the solvent are present in a ratio of about 1:16.8 (w/w). The term “about” as used herein means 10 percent more or 10 percent less than the denoted value.

Accordingly, step (c) of the herein described methods may comprise adding further solvent so that the flower material and the solvent are present in a ratio of about 1:16.8 (w/w) in the mixture of the flower material and the solvent.

In other words, step (c) of the herein described methods may comprise adding further solvent thereby obtaining a mixture of the flower material and solvent in a ratio of about 1:16.8 (w/w).

In general, the skilled person knows how proper plant material to solvent ratios are determined. For example, if one would use grounded or comminuted plant or flower material, the material would have a higher bulk density and less extraction solvent would be necessary.

Preferably after further solvent is added percolation is performed. Said step is herein referred to as “percolation step”. The “maceration step” may already be performed in a percolator. If the “maceration step” is not performed in a percolator the mixture of the flower material and the solvent is transferred into a percolator prior to the “percolation step”.

Then the solvent is allowed to flow through the flower material. In other words, the solvent is seeped through the flower material. The flow rate may be in the range of 1.0 ml/min to 10.0 ml/min per kg flower material, preferably 3.55 to 5.45 ml/min per kg flower material, more preferably 4.0 ml/min to 5.0 ml/min per kg flower material (i.e. 4.1, 4.2, 4.3, 4.4, 4.5, 4.6, 4.7, 4.8, 4.9 or 5.0 ml/min per kg flower material), most preferably 4.0 ml/min per kg flower material. The solvent may be pumped and allowed to flow several times through the flower material. It is preferred that the flower material is constantly covered with solvent. Percolation continues until the percolate is recovered. The percolation may be performed for 1 to 400 h, preferably, 63 to 97 hours (i.e. 63 h, 64 h, 65 h, 66 h, 67 h, 68 h, 69 h, 70 h, 71 h, 72 h, 73 h, 74 h, 75 h, 76 h, 77 h, 78 h, 79 h, 80 h, 81 h, 82 h, 83 h, 84 h, 85 h, 86 h, 87 h, 88 h, 89 h, 90 h, 91 h, 92 h, 93 h, 94 h, 95 h, 96 h, or 97 h), most preferably 86 hours (h). The percolate is the solvent with the extracted substances from the Cannabis flower material, i.e. the solvent with the dissolved e.g. cannabinoids (preferably THC) and/or other (pharmaceutically active) substances (e.g. terpenes). The solvent with the dissolved e.g. cannabinoids (preferably THC) and/or other (pharmaceutically active) substances (e.g. terpenes) is also referred to as miscella.

The solvent with the dissolved THC recovered from the percolator may already be the Cannabis plant extract. Said Cannabis plant extract may be further concentrated and then referred to as Cannabis plant soft extract. However, the solvent with the dissolved THC recovered from the percolator may be subjected to additional purification and/or separation steps as described further below.

Additional information regarding percolation can be found in European pharmacopeia (04/2019:0765).

It is envisaged that the e.g. cannabinoids (preferably THC) and/or other (pharmaceutically active) substances (e.g. terpenes, terpenoids (aroma), further secondary flower substances such as chlorophylls and flavonoids) from the Cannabis flower material dissolve in the solvent during the “maceration step” and “percolation step”. In other words, THC is extracted from the Cannabis flower material during the “maceration step” and “percolation step”.

Accordingly, the extraction of dried Cannabis flower material may be performed as follows:

The dried flowers which are only trimmed prior to the drying step must have a minimum content of delta-9-THC equivalents (delta-9-THC+delta-9-THCA*0.877) of 10 wt % by dry weight (i.e. at least 10 percent by dry weight (w/w)). An amount of 16.7 kg (15.9 kg to 17.5 kg) of ethanol 96% (v/v) of pharmaceutical grade is used per kg dried Cannabis flower material. The extraction process is performed by exclusion of light at 15° C. to 25° C. in a stainless-steel extraction vessel of cylindric shape and of pharmaceutical grade equipped with a strainer at the bottom. The extraction is subdivided in maceration (static extraction) and percolation (mobile extraction). The dried Cannabis flower material is first soaked with half the amount of ethanol (8.35 kg±5% per kg flowers) for 24 h±1 h during maceration which enables to dissolve most of delta-9-THC and other cannabinoids as well as related secondary plant substances like terpenes (aroma), fats and chlorophylls. After maceration, the second half amount of ethanol is added to the miscella (i.e. the solvent with the dissolved e.g. cannabinoids (e.g. THC and/or CBD) and/or other (pharmaceutically active) substances (e.g. terpenes)), and the percolation is started.

Thereby, the miscella is seeped through the flower material with a flow rate of 4.0 to 5.0 ml/min per kg flowers for 63 to 97 h and collected in a mobile buffer tank made of pharmaceutical stainless-steel through the strainer in the extraction vessel. The flower material is laying on a strainer and the opening from which the extract flows into the buffer tank is located below the strainer. In addition, the extract is pumped through a pharmaceutical grad filter on the way to the buffer/collection tank. Said collected miscella (i.e. the solvent with the dissolved e.g. cannabinoids (e.g. THC and/or CBD) and/or other (pharmaceutically active) substances (e.g. terpenes)) may be the Cannabis plant extract but usually has not the desired THC concentration. Accordingly, it is preferred that the miscella is subjected to further concentration and/or purification and/or separation steps and may then be referred to as Cannabis plant soft extract.

The herein described methods may also comprise a step in which the solvent with the dissolved e.g. cannabinoids (preferably THC) and/or other (pharmaceutically active) substances (e.g. terpenes) is separated from the (Cannabis flower) plant material. Said step is herein referred to as “separation step”. When the solvent with the dissolved THC is separated from the Cannabis plant material said solvent is referred to as Cannabis plant extract. Accordingly, in the “separation step” the (Cannabis) plant extract is separated from the mixture of the plant material, preferably, Cannabis plant material, more preferably, flower material of a Cannabis plant and the solvent. Accordingly, step (c) of the herein described methods may comprise separating the (Cannabis) plant extract from the mixture of the plant material, preferably, Cannabis plant material, more preferably, flower material of a Cannabis plant and the solvent.

In other words, step (c) of the herein described methods may comprise separating the Cannabis plant extract (preferably comprising delta-9-tetrahydrocannabinol (THC)) from the flower material.

In the “separation step” the skilled person may apply all suitable methods to separate liquids from solid material such as centrifugation, filtration, decanting and distillation.

As mentioned above when percolation is performed the percolator may contain a strainer which separates Cannabis flower material from the solvent. However, the strainer of the percolator may only remove larger parts of the Cannabis flower material so that an additional purification and/or separation steps may be applied. The additional purification and/or separation step may be filtration.

Preferably, the solvent with the dissolved THC is further purified by filtration through a polypropylene-based particle filter of pharmaceutical grade and a deposition rate of 1.5 μm. Preferably, the operating pressure and temperature should not exceed 5 bar and 50° C., respectively. As for all herein described methods the skilled person is readily capable of measuring parameters such as temperature and pressure via standard techniques. The filtration can be performed either in between percolation and collection in the mobile buffer tank or afterwards by collecting the filtered miscella in a second mobile buffer tank of the same stainless-steel grade.

In context of the invention a preferred method for producing a Cannabis plant extract comprising delta-9-tetrahydrocannabinol (THC) from a Cannabis plant comprises the following steps:
(a) providing a Cannabis plant which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract comprising delta-9-tetrahydrocannabinol (THC) from the flower material;
wherein the treating step as defined in step (c) comprises the following steps:
(i) overlaying the flower material with a solvent in a ratio of about 1:8.4 (w/w) thereby obtaining a mixture of the flower material and the solvent;
(ii) performing maceration for about 24 h at a temperature of 15° C. to 25° C. and exclusion of light;
(iii) adding further solvent thereby obtaining a mixture of the flower material and solvent in a ratio of about 1:16.8 (w/w);
(iv) performing percolation, wherein the solvent is seeped through the flower material with a flow rate of 3.55 to 5.45 ml/min per kg flower material, preferably 4.0 ml/min per kg flower material for 63 to 97 h, preferably for 86 h at a temperature of 15° C. to 25° C. and exclusion of light; and
(v) separating the Cannabis plant extract from the Cannabis flower material.

The invention also relates to a method for producing a Cannabis plant extract comprising delta-9-tetrahydrocannabinol (THC) from a Cannabis plant comprising the following steps:
(a) providing a Cannabis plant which comprises delta-9-tetrahydrocannabinol (THC) in an amount of at least 10 percent by dry weight;
(b) trimming and drying the flower material separated from the remaining plant material; and
(c) treating the flower material of step (b) with a solvent and separating the Cannabis plant extract comprising delta-9-tetrahydrocannabinol (THC) from the flower material;
wherein the treating step as defined in step (c) comprises the following steps:
(i) overlaying the flower material with a solvent in a ratio of about 1:8.4 (w/w) thereby obtaining a mixture of the flower material and the solvent;
(ii) performing maceration for about 24 h at a temperature of 15° C. to 25° C. and exclusion of light;
(iii) adding further solvent thereby obtaining a mixture of the flower material and solvent in a ratio of about 1:16.8 (w/w);
(iv) performing percolation, wherein the solvent is seeped through the flower material with a flow rate of 3.55 to 5.45 ml/min per kg flower material, preferably 4.0 ml/min per kg flower material for 63 to 97 h, preferably for 86 h at a temperature of 15° C. to 25° C. and exclusion of light; and
(v) separating the Cannabis plant extract from the Cannabis flower material.

The invention also relates to a method for producing a Cannabis plant extract comprising delta-9-tetrahydrocannabinol (THC) from a Cannabis plant comprising the following steps:
(a) providing a Cannabis plant of the Cannabis variety DKJ127;
(b) trimming and drying the flower material separated from the remaining plant material; and
(c) treating the flower material of step (b) with a solvent and separating the Cannabis plant extract comprising delta-9-tetrahydrocannabinol (THC) from the flower material.

The concentration/amount of THC in the Cannabis plant used in the herein described methods (preferably the variety DKJ127) may be determined to assess whether the concentration/amount of THC is sufficient, e.g. that the Cannabis plant extract produced by the methods described herein will have the desired concentration/amount of THC.

Accordingly, the invention also relates to a method for producing a Cannabis plant extract comprising delta-9-tetrahydrocannabinol (THC) from a Cannabis plant comprising the following steps:
(a) providing a Cannabis plant of the Cannabis variety DKJ127 which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract comprising delta-9-tetrahydrocannabinol (THC) from the flower material.

In context of the invention a preferred method for producing a Cannabis plant extract comprising delta-9-tetrahydrocannabinol (THC) from a Cannabis plant comprises the following steps:
(a) providing a Cannabis plant of the Cannabis variety DKJ127;
(b) trimming and drying the flower material separated from the remaining plant material; and
(c) treating the flower material of step (b) with a solvent and separating the Cannabis plant extract comprising delta-9-tetrahydrocannabinol (THC) from the flower material;
wherein the treating step as defined in step (c) comprises the following steps:
(i) overlaying the flower material with a solvent in a ratio of about 1:8.4 (w/w) thereby obtaining a mixture of the flower material and the solvent;
(ii) performing maceration for about 24 h at a temperature of 15° C. to 25° C. and exclusion of light;
(iii) adding further solvent thereby obtaining a mixture of the flower material and solvent in a ratio of about 1:16.8 (w/w);
(iv) performing percolation, wherein the solvent is seeped through the flower material with a flow rate of 3.55 to 5.45 ml/min per kg flower material, preferably 4.0 ml/min per kg flower material for 63 to 97 h, preferably for 86 h at a temperature of 15° C. to 25° C. and exclusion of light; and
(v) separating the Cannabis plant extract from the Cannabis flower material.

As mentioned above a solvent is used to “remove” the desired substance (e.g. cannabinoids (e.g. THC and/or CBD) and/or other (pharmaceutically active) substances (e.g. terpenes)) from the plant material, preferably, Cannabis plant material. Any solvent may be used as long as the solvent is capable of “removing” the desired substance from the plant material. In other words, the desired substance should be soluble or dissolvable in the used solvent.

Non-limiting examples of solvents that may be suitable are alcohols (e.g. methanol, ethanol, propanol, butanol, propylene glycol etc.), water, hydrocarbons (e.g. butane, hexane, etc.), polar organic solvents (e.g. ethyl acetate, polyethylene glycol, etc.) or a supercritical fluid (e.g. liquid CO₂) as well as aqueous solutions thereof.

Suitable non-polar solvents may be C5-C12 straight chain or branched chain alkanes, C1-C12 alcohols or carbonate esters of C1-C12 alcohols. The more volatile solvents may be particularly useful, as they are more easily removed from the extract if desired. Although completely evident for the skilled person it is pointed out that also mixtures of the mentioned solvents may be used for the herein described methods. Preferred solvents for the herein described methods are ethanol, butanol, alkanes (such as pentane, heptane and propane), ethyl ether, tert butyl-methyl-ether, methyl-ethyl-ketone, acetone, ethyl acetate, CO₂. A very preferred solvent for the herein described methods is ethanol, in particular 96 Vol-% ethanol. In the context of the present invention solvents of pharmaceutical grade are preferably used.

Accordingly, the invention relates to a method for producing a Cannabis plant extract comprising delta-9-tetrahydrocannabinol (THC) from a Cannabis plant comprising the following steps:
(a) providing a Cannabis plant which comprises delta-9-tetrahydrocannabinol (THC) in a sufficient amount;
(b) trimming and drying the flower material separated from the remaining plant material; and
(c) treating the flower material of step (b) with ethanol and separating the Cannabis plant extract comprising delta-9-tetrahydrocannabinol (THC) from the flower material.

In context of the invention a preferred method for producing a Cannabis plant extract comprising delta-9-tetrahydrocannabinol (THC) from a Cannabis plant comprises the following steps:
(a) providing a Cannabis plant which comprises delta-9-tetrahydrocannabinol (THC) in a sufficient amount;
(b) trimming and drying the flower material separated from the remaining plant material; and
(c) treating the flower material of step (b) with ethanol and separating the Cannabis plant extract comprising delta-9-tetrahydrocannabinol (THC) from the flower material;
wherein the treating step as defined in step (c) comprises the following steps:
(i) overlaying the flower material with ethanol in a ratio of about 1:8.4 (w/w) thereby obtaining a mixture of the flower material and the ethanol;
(ii) performing maceration for about 24 h at a temperature of 15° C. to 25° C. and exclusion of light;
(iii) adding further ethanol thereby obtaining a mixture of the flower material and ethanol in a ratio of about 1:16.8 (w/w);
(iv) performing percolation, wherein the ethanol is seeped through the flower material with a flow rate of 3.55 to 5.45 ml/min per kg flower material, preferably 4.0 ml/min per kg flower material for 63 to 97 h, preferably for 86 h at a temperature of 15° C. to 25° C. and exclusion of light; and
(v) separating the Cannabis plant extract from the Cannabis flower material.

In context of the invention a particular preferred method for producing a Cannabis plant extract comprising delta-9-tetrahydrocannabinol (THC) from a Cannabis plant comprises the following steps:
(a) providing a Cannabis plant of the Cannabis variety DKJ127;
(b) trimming and drying the flower material separated from the remaining plant material; and
(c) treating the flower material of step (b) with ethanol and separating the Cannabis plant extract comprising delta-9-tetrahydrocannabinol (THC) from the flower material;
wherein the treating step as defined in step (c) comprises the following steps:
(i) overlaying the flower material with ethanol in a ratio of about 1:8.4 (w/w) thereby obtaining a mixture of the flower material and the ethanol;
(ii) performing maceration for about 24 h at a temperature of 15° C. to 25° C. and exclusion of light;
(iii) adding further ethanol thereby obtaining a mixture of the flower material and ethanol in a ratio of about 1:16.8 (w/w);
(iv) performing percolation, wherein the ethanol is seeped through the flower material with a flow rate of 3.55 to 5.45 ml/min per kg flower material, preferably 4.0 ml/min per kg flower material for 63 to 97 h, preferably for 86 h at a temperature of 15° C. to 25° C. and exclusion of light; and
(v) separating the Cannabis plant extract from the Cannabis flower material.

It is further envisaged herein that the Cannabis plant extract produced according to the methods described herein is further concentrated by a concentration step. “Concentration step” in this context means that solvent is removed from liquid Cannabis plant extract. The solvent may be removed via evaporation or freeze-drying. However, the concentration step (e.g. evaporation step) may also be part of the methods according to the invention and described herein. Thus, the herein described methods may further comprise evaporating the solvent from the liquid Cannabis plant extract to concentrate the extract.

Additionally, a decarboxylation step may also be part of the methods of the invention. It is envisaged herein that after the concentration step and/or decarboxylation step the Cannabis plant extract may be referred to as Cannabis plant soft extract.

Accordingly, the invention relates to a method for producing a Cannabis plant extract comprising delta-9-tetrahydrocannabinol (THC) from a Cannabis plant comprising the following steps:
(a) providing a Cannabis plant which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract comprising delta-9-tetrahydrocannabinol (THC) from the flower material;
wherein the method further comprises evaporating the solvent from the liquid Cannabis plant extract to concentrate the extract.

Preferably, the solvent in the Cannabis plant extract is evaporated in vacuum by using a rotary evaporator (e.g. Manufacturer: Büchi; Model: Rotavapor R-220 Pro; Distillation rate: up to 12 liters ethanol/h; Flask size: 20 liters; Maximum capacity of the flask: 12 liters; Continuous feed).

The water bath temperature, the pressure, and the rotation speed may be about 72° C. (i.e. 67° C., 68° C., 69° C., 70° C., 71° C., 72° C., 73° C., 74° C., 75° C., 76° C., 77° C. or a fraction thereof), about 185 mbar (i.e. 175 mbar, 176 mbar, 177 mbar, 178 mbar, 179 mbar, 180 mbar, 181 mbar, 182 mbar, 183 mbar, 184 mbar, 185 mbar, 186 mbar, 187 mbar, 188 mbar, 189 mbar, 190 mbar, 191 mbar, 192 mbar, 193 mbar, 194 mbar, 195 mbar or a fraction thereof), and about 150 rpm (i.e. 145 rpm, 146 rpm, 147 rpm, 148 rpm, 149 rpm, 150 rpm, 151 rpm, 152 rpm, 153 rpm, 154 rpm, 155 rpm, 156 rpm, 157 rpm, 158 rpm, 159 rpm or 160 rpm) respectively. These conditions lead to a steam temperature of ethanol of 41° C.-42° C. and an evaporation performance of approximately 10 L/h. The evaporation may be concluded when the steam temperature drops below 30° C. indicating the almost full removal of ethanol. The overall evaporation time can be kept constant independently on the batch size by parallelly evaporating the solvent with several rotary evaporators. An almost solvent-free Cannabis plant extract of dark green to dark brown resin-type nature is obtained mainly consisting of delta-9-THCA and partially activated delta-9-THC.

In the Cannabis plant, THC occurs mainly as tetrahydrocannabinolic acid (THCA). Therefore, it may be beneficial or desired to convert the tetrahydrocannabinolic acid in the Cannabis plant extract produced according to the herein described methods into delta-9-tetrahydrocannabinol (THC). The conversion of tetrahydrocannabinolic acid (THCA) into delta-9-tetrahydrocannabinol (THC) is a decarboxylation. Decarboxylation is a chemical reaction that releases carbon dioxide. The conversion step (i.e. the decarboxylation step) may also be part of the methods according to the invention and described herein. Thus, the herein described methods may further comprise heating the Cannabis plant extract to a certain temperature to decarboxylate tetrahydrocannabinolic acid (THCA) to delta-9-tetrahydrocannabinol (THC).

To decarboxylate tetrahydrocannabinolic acid (THCA) to delta-9-tetrahydrocannabinol (THC) the Cannabis plant extract may be heated to a temperature of 50° C. to 150° C., preferably about 80° C. (i.e. 75° C., 76° C., 77° C., 78° C., 79° C., 80° C., 81° C., 82° C., 83° C., 84° C., 85° C. or any fraction thereof). Preferably, the decarboxylation is performed under vacuum. The vacuum is preferably about 185 mbar (i.e. 175 mbar, 176 mbar, 177 mbar, 178 mbar, 179 mbar, 180 mbar, 181 mbar, 182 mbar, 183 mbar, 184 mbar, 185 mbar, 186 mbar, 187 mbar, 188 mbar, 189 mbar, 190 mbar, 191 mbar, 192 mbar, 193 mbar, 194 mbar, 195 mbar or a fraction thereof). The decarboxylation may be performed for about 72 h (i.e. 67 h, 68 h, 69 h, 70 h, 71 h, 72 h, 73 h, 74 h, 75 h, 76 h, 77 h or any fraction thereof).

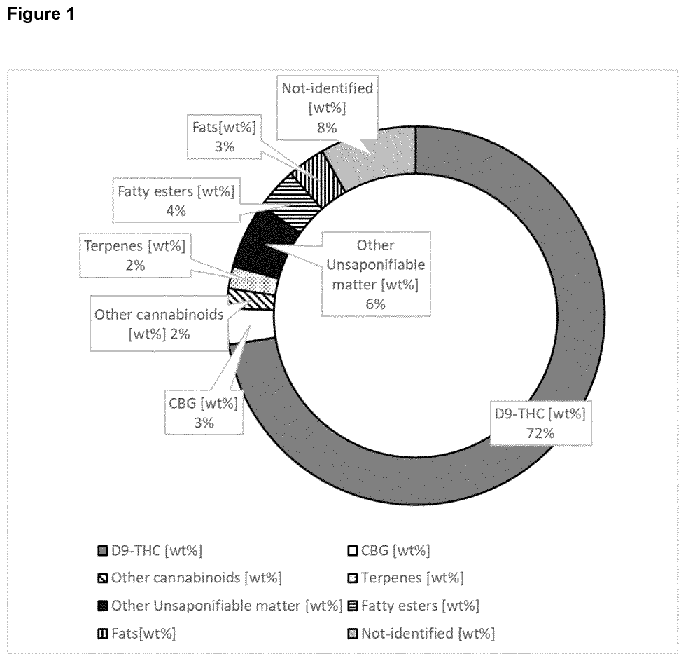

It is envisaged herein that after the concentration step and the decarboxylation step the Cannabis plant extract may be referred to as Cannabis plant soft extract and has components as shown in FIG. 1 and Tables 1 to 7.

TABLE 1
Overview of characterization of soft extract

| Substance and Substance classes | Soft extract |
|---|---|
| delta-9-THC [wt %] | 72.25 |
| CBG [wt %] | 3.08 |
| Other cannabinoids [wt %] | 1.67 |
| Terpenes [wt %] | 1.88 |
| Flavonoids/Phytosterols/Tocopherols/Coumarins [wt %] | 0.01 |
| Other Unsaponifiable matter [wt %] | 5.66 |
| Residual solvents [wt %] | 0.40 |
| Fatty esters [wt %] | 3.64 |
| Fats [wt %] | 3.24 |
| Not-identified [wt %] | 8.19 |

TABLE 2
Overview of cannabinoids in soft extract.

| Substances | Soft extract/wt % |
|---|---|
| THCA | 0.14 |
| delta-9-THC | 72.25 |
| CBG | 3.08 |
| CBGA | 0.28 |
| CBN | 0.57 |
| CBDA | 0.05 |
| CBD | 0.19 |
| CBC | 0.43 |
| delta-8-THC | <0.05 |
| THCV | 0.27 |
| Sum of cannabinoids without D9-THC and CBG | 1.67 |

TABLE 3
Overview of terpenes and terpenoids in soft extract.

| Substances | Soft extract/mg/g |
|---|---|
| α-Pinene | 0.048 |
| Camphene | 0.008 |
| β-Pinene | 0.033 |
| β-Myrcene | 0.145 |
| δ-3-Caren | <0.01 |
| α-Terpinene | 0.013 |
| p-Cymen | 0.000 |
| Limonene | 0.273 |
| Eucalyptol | 0.023 |
| Ocimene | 0.080 |
| γ-Terpinene | 0.022 |
| Terpinolene | 0.013 |
| Linalool | 1.055 |
| Isopulegol | <0.01 |
| Geraniol | <0.01 |
| β-Caryophyllene | 5.200 |
| α-Humulene | 1.775 |
| Nerolidol | 2.150 |
| Caryophyllenoxide | 0.398 |
| Guaiol | 4.875 |
| α-Bisabolol | 2.700 |
| Sum of terpenes and terpenoids/mg/g | 18.81 |
| Sum of terpenes and terpenoids/wt % | 1.88 |

TABLE 4
Overview of flavonoids in soft extract.

| Substances | Soft extract/μg/kg |
|---|---|
| Kaempferol | 210 |
| Quercetin | <1 |
| Orientin | 72 |
| Vitexin | 180 |
| Luteolin-7-Glucosid | 69 |
| Isovitexin | 8.9 |
| Apiin | 1.4 |
| Cannflavin A | 6900 |
| Cannflavin B | 2700 |
| Apigenin | 180 |
| Hyperosid | 74 |
| Isoquercitin | 56 |
| Luteolin | 130 |
| Myricetin | <1 |
| Rutoside | <1 |
| Sum of flavonoids/μg/kg | 10581 |
| Sum of flavonoids/wt % | 1.06 · 10⁻³ |

TABLE 5
Overview of phytosterols in soft extract.

| Substances | Soft extract/μg/g |
|---|---|
| beta-sitosterol | 38.93 |
| Campesterol | 5.08 |
| Stigmasterol | 19.04 |
| Sum of flavonoids/μg/g | 63.1 |
| Sum of flavonoids/wt % | 6.31 · 10⁻³ |

TABLE 6
Overview of α-Tocopherol in soft extract.

| Substances | Soft extract/μg/g |
|---|---|
| α-Tocopherol/μg/g | 11.78 |
| α-Tocopherol/wt % | 1.18 · 10⁻³ |

TABLE 7
Overview of unsaponifiable and saponifiable matter in soft extract.

| Substances | Soft extract | |
|---|---|---|
| Unsaponifiable matter/wt % | 84.4 | wt % |
| Sum of unsaponifiable matter from 1-6 | 78.9 | wt % |
| Other unsaponifiable matter | 5.5 | wt % |

| | |
|---|---|
| Saponification value | 12 |
| Ester value | 5.5 |
| Average weight of fatty esters/wt % | 3.64 |
| Acid value | 6.5 |
| Weight of fatty acids/wt % | 3.24 |

Accordingly, the invention relates to a method for producing a Cannabis plant extract comprising delta-9-tetrahydrocannabinol (THC) from a Cannabis plant comprising the following steps:
(a) providing a Cannabis plant which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract comprising delta-9-tetrahydrocannabinol (THC) from the flower material;
wherein the method further comprises heating the Cannabis plant extract to a temperature of about 80° C. under vacuum at about 185 mbar to decarboxylate tetrahydrocannabinolic acid (THCA) to delta-9-tetrahydrocannabinol (THC).

It is envisaged herein that at least 90% to 100% of the THCA in the Cannabis plant extract is decarboxylated to THC (i.e. 90%, 91%, 92%, 93%, 94%, 95%, 96%, 97%, 98%, 99% or 100%, or any fraction thereof, e.g. 95.7%).

The full transformation of delta-9-THCA to delta-9-THC is preferably conducted via thermally induced decarboxylation in vacuum. Therefore, the rotary evaporator is used by adjusting the water bath temperature, pressure, and rotation to 80° C., 185 mbar and 150 rpm, respectively. The decarboxylation time may be 72 h. The resulting Cannabis plant extract has a dark green to dark brown resin-type nature with an average THC content of about 70 to 74 wt. %. The conversion factor between dried flowers and soft extracts amounts to 4.7 on average. A conversion factor of 4.7 means that 25 kg of cannabis flowers would results in roughly 5.3 kg Cannabis plant soft extract when obtained by the methods described herein.

It is also envisaged herein that the methods according to the invention further comprise evaporating the solvent from the liquid Cannabis plant extract to concentrate the extract and heating the Cannabis plant extract to a certain temperature to decarboxylate tetrahydrocannabinolic acid (THCA) to delta-9-tetrahydrocannabinol (THC).

Accordingly, the invention relates to a method for producing a Cannabis plant (soft) extract comprising delta-9-tetrahydrocannabinol (THC) from a Cannabis plant comprising the following steps:
(a) providing a Cannabis plant which comprises delta-9-tetrahydrocannabinol (THC) in a sufficient amount;
(b) trimming and drying the flower material separated from the remaining plant material;
(c) treating the flower material of step (b) with a solvent and separating the Cannabis plant extract comprising delta-9-tetrahydrocannabinol (THC) from the flower material;
(d) evaporating the solvent from the liquid Cannabis plant extract to concentrate the extract; and
(e) heating the Cannabis plant extract to a temperature of about 80° C. under vacuum at about 185 mbar to decarboxylate tetrahydrocannabinolic acid (THCA) to delta-9-tetrahydrocannabinol (THC).

As mentioned above, the herein described methods obviate the need for a winterization step. Accordingly, it is envisaged that the herein described methods are performed without a winterization step. “Winterization step” or “winterization” is referred to herein to method steps in which the temperature of a Cannabis plant extract is lowered in particular to remove e.g. waxes. Winterization is frequently performed after the Cannabis plant extract has been concentrated e.g. by vaporization. Usually during winterization the temperature is lowered below 10° C., preferably below 0° C. such as −5, −10, −20 or −25° C. Then the Cannabis plant extract is usually incubated at the low temperature for e.g. 48 h. After the incubation step, the precipitate is removed e.g. via centrifugation or filtration. Typically before the temperature of the Cannabis plant extract is lowered said extract is mixed with (additional) organic solvent such es methanol or ethanol. Winterization is described e.g. in DE 103 37 458 A1 and in WO 2002/064109 A2.

Accordingly, the present invention relates to a method for producing a Cannabis plant (soft) extract comprising delta-9-tetrahydrocannabinol (THC) from a Cannabis plant wherein the method does not comprise a winterization step. The phrases “wherein the method does not comprise a winterization step” and “does not involve a winterization step” is used synonymously with the phrase “no winterization step is performed”. Accordingly, the invention relates to a method for producing a Cannabis plant (soft) extract comprising delta-9-tetrahydrocannabinol (THC) from a Cannabis plant wherein no winterization step is performed.

Accordingly, the invention relates to a method for producing a Cannabis plant extract comprising delta-9-tetrahydrocannabinol (THC) from a Cannabis plant comprising the following steps:
(a) providing a Cannabis plant which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract comprising delta-9-tetrahydrocannabinol (THC) from the flower material,
wherein the method does not involve a winterization step.

The invention further relates to a method for producing a Cannabis plant extract comprising the following steps:
(a) providing a Cannabis plant, preferably which comprises delta-9-tetrahydrocannabinol (THC) in a sufficient amount;
(b) trimming and drying the flower material separated from the remaining plant material; and
(c) treating the flower material of step (b) with a solvent and separating the Cannabis plant extract from the mixture of the flower material and the solvent;
wherein the treating step as defined in step (c) comprises the following steps:
(i) overlaying the flower material with a solvent in a ratio of about 1:8.4 (w/w) thereby obtaining a mixture of the flower material and the solvent;
(ii) performing maceration for about 24 h at a temperature of 15° C. to 25° C. and exclusion of light;
(iii) adding further solvent thereby obtaining a mixture of the flower material and solvent in a ratio of about 1:16.8 (w/w);
(iv) performing percolation, wherein the solvent is seeped through the flower material with a flow rate of 3.55 to 5.45 ml/min per kg flower material, preferably 4.0 ml/min per kg flower material for 63 to 97 h, preferably for 86 h at a temperature of 15° C. to 25° C. and exclusion of light; and
(v) separating the Cannabis plant extract from the Cannabis flower material,
wherein the method does not involve a winterization step.

The invention also relates to the plant extract produced by/obtainable by the methods and processes disclosed herein. Thus, the invention also relates to the Cannabis plant extract produced by/obtainable by the methods described herein. Accordingly, the invention also relates to the Cannabis plant extract comprising delta-9-tetrahydrocannabinol (THC) produced by/obtainable by the methods described herein.

Accordingly, the present invention relates to a Cannabis plant extract comprising delta-9-tetrahydrocannabinol (THC) as obtainable by the methods described herein. Said Cannabis plant extract may be in a liquid form. Accordingly, the invention relates to a liquid Cannabis plant extract comprising delta-9-tetrahydrocannabinol (THC) as obtainable by the methods described herein. As also described elsewhere herein the Cannabis plant extract comprising delta-9-tetrahydrocannabinol (THC) as obtainable by the methods described herein may be in a solvent-free and decarboxylated form. Accordingly, the present invention relates to a Cannabis plant extract comprising delta-9-tetrahydrocannabinol (THC) as obtainable by the methods described herein in solvent-free and decarboxylated form.

The present invention also relates to a Cannabis plant extract as obtainable by the methods described herein comprising delta-9-tetrahydrocannabinol (THC) in an amount of about 70 to 74 percent by weight of the Cannabis plant extract. The term “about” as used in the context of the THC amount means 20 percent more or 20 percent less than the denoted value. In the context of the present invention, the THC amount of any particular Cannabis plant as described herein may be qualitatively and quantitatively determined using methods well known to those skilled in the art, such as Thin Layer Chromatography (TLC) or High Performance Liquid Chromatography (HPLC).

In the context of the present invention, the Cannabis plant extract, preferably the Cannabis plant soft extract as produced by/obtainable by the methods described herein may be composited as shown in FIG. 1 and Tables 1 to 7.

It is envisaged herein that the Cannabis plant extract or Cannabis plant soft extract as obtainable by the herein described methods may be further processed to a composition comprising desired substances of the Cannabis plant, e.g. delta-9-tetrahydrocannabinol (THC) and/or terpenes. However, it is also envisaged that the Cannabis plant extract as obtainable by the herein described methods is directly used (i.e. without further processing) for e.g. pharmaceutical or formulations. A Cannabis plant extract that was not further processed may be referred to as native extract. Accordingly, the term “a composition comprising desired substances of the Cannabis plant, e.g. delta-9-tetrahydrocannabinol (THC) and/or terpenes as obtainable by the herein described methods” as used herein can refer to the Cannabis plant extract as obtainable by the herein described methods but it can also refer to the Cannabis plant extract as obtainable by the herein described methods that has been further processed. Accordingly, “a composition as obtainable by the herein described methods” may be used synonymously with “a Cannabis plant (soft) extract as obtainable by the herein described methods”. In other words, it is envisaged herein that the Cannabis plant extract as obtainable by the herein described methods is directly used for a pharmaceutical formulation, i.e. directly formulated in a pharmaceutical or formulation or that the Cannabis plant extract as obtainable by the herein described methods is first further processed into a composition and said composition is then used for a pharmaceutical formulation. The processing of the Cannabis plant extract as obtainable by the herein described methods into a composition may be necessary to allow e.g. storage and/or transport of said Cannabis plant extract.

The Cannabis plant extract may be processed e.g., without limitation, by changing the pH or by adding one or more solvents in a preferred concentration. In some instances, the extract as described herein may also be filtered to remove particulate material, for example, by passing the extract through filter paper or a fine sieve with pore sizes suitable for filtration. As such, the composition of the present invention is preferably provided in liquid form. As will be appreciated, one or more additional compounds (e.g. cannabinoid, terpene or terpenoid compounds) may be added to the composition as described herein. The addition of compounds may be to compensate for natural variations in the relative amounts of certain compounds being expressed by the Cannabis plant which provides the extract. The added compounds may be natural or synthetic versions of the desired compound(s)

However, it is also envisaged that the Cannabis plant extract as obtainable by the herein described methods itself, i.e. the native extract may be used as the pharmaceutical formulation.

Accordingly, everything that is disclosed herein for the composition and the pharmaceutical formulation applies also to the Cannabis plant extract or Cannabis plant soft extract and vice versa. When it is referred herein to “the Cannabis plant (soft) extract” is it is obvious for the skilled person that also reference to “the Cannabis plant (soft) extract as obtainable by the herein described methods” is intended.

The Cannabis plant extract or Cannabis plant soft extract as obtainable by the herein described methods may be analysed by methods known in the art.

The Cannabis plant soft extract may be visually inspected to define the color which may be yellow to green.

The extract may be further analysed via TLC according to Ph.Eur. 2.2.27 and according to DAB monograph “Eingestellter Cannabisextrakt”. The extract may be further analysed via HPLC according to Ph.Eur. 2.2.29 and according to internal validated method on the basis of the DAB monograph “Eingestellter Cannabisextrakt” by comparing the cannabinoids profile with retention times of reference standards and by a reference chromatogram of this product.

The extract may be further analysed via GC according to Ph.Eur. 2.2.28 and according to internal validated GC-MS method by comparing the terpenes/terpenoids profile with retention times of reference standards and by a reference chromatogram of this product. The water content may be determined according to Ph.Eur. 2.5.12 (Karl Fischer) and the ethanol content may be analysed according to Ph.Eur. 2.2.28 and according to internal validated GC method. Microbial impurities may be analysed according to Ph.Eur. 5.1.4-2 including Ph.Eur. 2.6.12 and 2.6.31.

The Cannabis plant extract or Cannabis plant soft extract as obtainable by the herein described methods may be analysed for waxes as follows:

1.00 kg soft extract may be mixed with 1.75 kg of ethanol and stirred for 30 minutes. The obtained solution may be filtered through a 71 μm metal mesh sieve and Ethanol may be added to obtain a soft extract to ethanol solution with a ratio of 1:2.3. The solution may be placed in a freezer for 48 hour (winterization). After 48 hours there may be no significant fatty layer on the top, only a few small streaks of opaque plaques/strings may be visible. Filtration using a 50 μm and second 21 μm metal mesh sieve may be performed. A dark fatty material may be collected. The collected dark fatty material may be solved in about 30 ml ethanol and centrifuged. When e.g. 20 grams of waxes are obtained the wax content of the Cannabis plant extract is about 2%.

The Cannabis plant extracts as obtainable according to the herein described methods are preferably substantially free or have low levels of waxes and other non-specific lipid soluble material but preferably contain substantially all of the cannabinoids naturally present in the plant, most preferably in substantially the same ratios in which they occur in the intact Cannabis plant. In other words, Cannabis plant extracts as obtainable according to the herein described methods are preferably substantially free or have low levels of saponifiable substances. Saponifiable substances as used herein may refer to lipids including free fatty acids, triglycerides and waxes (mono-esterified fatty acids). The Cannabis plant extracts as obtainable according to the herein described methods comprises preferably less than 10%, 9%, 8%, 7%, 6%, 5%, 4%, 3%, 2% or 1% saponifiable substances by weight, most preferably less than 8% saponifiable substances by weight. The Cannabis plant extract or Cannabis plant soft extract as obtainable according to the herein described methods comprises preferably less than 3%, 2.1% or 2% waxes by weight.

As mentioned above the Cannabis plant extracts as obtainable according to the herein described methods preferably comprises THC.

Accordingly, it is envisaged that the composition (i.e. the Cannabis plant extract or Cannabis plant soft extract) comprises THC as main cannabinoid, however, the optional presence of other cannabinoids including, without limitation, cannabidiol (CBD), D-8-Tetrahydrocannabinol (delta-8-THC), cannabidiolic acid (CBDA) cannabichromene (CBC), tetrahydrocannabinolic acid (THCA), tetrahydrocannabivarin (THCV), cannabidivarin (CBDV), cannabinol (CBN), cannabigerolic acid (CBGA) and cannabigerol (CBG) is not excluded. Specifically, it will be appreciated that the present composition comprising THC as main cannabinoid may comprise low amounts of CBD, for example, less than 10% CBD by weight of the composition, or less than, 9%, 8%, 7%, 6%, 5% 4%, 3%, 2%, 1%, 0.5%, 0.2% or 0.1% CBD by weight of the composition, or may not comprise any measurable CBD. In the present invention, it is preferred that the composition comprises less than 0.5% CBD by weight, less than about 3.1% CBG by weight and less than about 2.5%, 2.2%, 2.1%, 2.0%, 1.9%, 1.8%, 1.7%, 1.6% or 1.5% other cannabinoids by weight of the composition, preferably less than about 1.7% other cannabinoids by weight of the composition. The entirety of cannabinoids, i.e. the cannabinoid fraction, typically accounts for the majority of the compounds present in the composition of the invention. It is preferred that the cannabinoid fraction, excluding THC as main cannabinoid, may be present in an amount of not more than about 6% by weight of the extract.

As already mentioned, the Cannabis plant extract as obtainable by the herein described methods may comprise THC. The Cannabis plant extract may comprise THC in an amount of at least about 60, 65, 70, 75 or 80 percent, but preferably, at least about 70 percent by weight of the Cannabis plant extract. Preferably, the Cannabis plant extract as obtainable by the herein described methods comprises THC in amount of about 70 to 74 (70, 71, 72, 73, 74 or any fraction thereof, e.g. 70.5, 71.5, 72.5, 73.5) percent by weight of the Cannabis plant extract. It is preferred that the Cannabis plant extract comprises THC in an amount of at least about 70 percent. Accordingly, the invention relates to a Cannabis plant extract as obtainable by the methods described herein comprising delta-9-tetrahydrocannabinol (THC) in an amount of at least about 70 percent by weight of the Cannabis plant extract. The term “about” as used in the context of the THC amount means 20 percent more or 20 percent less than the denoted value. In the context of the present invention, the THC amount of any particular Cannabis plant as described herein may be qualitatively and quantitatively determined using methods well known to those skilled in the art, such as Thin Layer Chromatography (TLC) or High Performance Liquid Chromatography (HPLC).

It is evident for the skilled person that during the herein described methods (particular during the maceration and or percolation step) not only THC but also other substances of the Cannabis plant are removed from the Cannabis plant material, preferably Cannabis flower material. In other words, besides THC other substances from the Cannabis flower material are dissolved in the solvent, preferably ethanol. As mentioned already herein it is envisaged that the Cannabis plant extract as obtainable by the herein described methods may additionally comprise terpenes and/or terpenoids. Non-limiting examples for terpenes are alpha-bisabolol, guaiol, beta-caryophyllene, alpha-pinene, camphene, beta-pinene, beta-myrcene, delta-3-carene, alpha-terpinene, p-cymene, limonene, eucalyptol, ocimene, gamma-terpinene, terpinolene, linalool, isopulegol, geraniol, alpha-humulene, nerolidol, caryophyllene oxide, terpineol, valencene, phellandrene, fenchol, borneol, phytol, sabinene, camphor, isoborneol, menthol, cedrene and/or squalene. Accordingly, the Cannabis plant extract as obtainable by the herein described methods may additionally comprise alpha-bisabolol, guaiol, beta-caryophyllene, alpha-pinene, camphene, beta-pinene, beta-myrcene, delta-3-carene, alpha-terpinene, p-cymene, limonene, eucalyptol, ocimene, gamma-terpinene, terpinolene, linalool, isopulegol, geraniol, alpha-humulene, nerolidol caryophyllene oxide, terpineol, valencene, phellandrene, fenchol, borneol, phytol, sabinene, camphor, isoborneol, menthol, cedrene and/or squalene. Preferably, the Cannabis plant extract as obtainable by the herein described methods comprises alpha-bisabolol, guaiol and/or beta-caryophyllene.

Furthermore, the Cannabis plant extract as obtainable by the herein described methods may additionally comprise flavonoids. Non-limiting examples of flavonoids are Kaempferol, Quercetin, Orientin, Vitexin, Apigenin, Hyperoside, Rutoside, Luteolin-7-Glucoside, Myricetin, Isovitexin, Isoquercitin, Apiin, Luteolin, Cannflavin A and Cannflavin B.

Furthermore, the Cannabis plant extract as obtainable by the herein described methods may additionally comprise phytosterols and/or vitamins. Non-limiting examples of phytosterols and/or vitamins are beta-sitosterol, campesterol, stigmasterol and alpha-tocopherol (Vitamin E).

The Cannabis plant extract as obtainable by the herein described methods may have the concentration of the above-mentioned substances in concentrations as shown in FIG. 1 and Tables 1 to 7.

Accordingly, it also envisaged that Cannabis plant extract as obtainable by the herein described methods comprises delta-9-tetrahydrocannabinol (THC) and terpenes and/or terpenoids. The Cannabis plant extract as obtainable by the herein described methods may comprise delta-9-tetrahydrocannabinol (THC) and a terpene selected from the group consisting of alpha-bisabolol, guaiol, and beta-caryophyllene. Accordingly, the invention relates to a Cannabis plant extract as obtainable by the herein described methods comprising delta-9-tetrahydrocannabinol (THC) and a terpene selected from the group consisting of alpha-bisabolol, guaiol, and beta-caryophyllene.

It is preferred that the Cannabis plant extracts as obtainable according to the herein described methods naturally comprises THC and alpha-bisabolol, guaiol and/or beta-caryophyllene. Accordingly, the Cannabis plant extract as obtainable according to the herein described methods is preferably an extract derived from/obtainable from a plant that does endogenously contain or produce THC and alpha-bisabolol, guaiol and/or beta-caryophyllene. Such plant extract may be derived from/obtainable from a plant that does endogenously contain or produce cannabinoids, cannabinoid-like substances and/or cannabimimetic compounds as well as terpenes and/or terpenoids provided that they are capable of producing THC and alpha-bisabolol, guaiol and/or beta-caryophyllene. Alternatively, the Cannabis plant extract as obtainable according to the herein described methods is an extract derived from/obtainable from a plant that is capable of producing cannabinoids, cannabinoid-like substances or cannabimimetic compounds as well as terpenes and/or terpenoids, but does not endogenously contain or produce them. Accordingly, the Cannabis plant extract as obtainable according to the herein described methods may also be derived from a transgenic plant, capable of heterologously producing THC and alpha-bisabolol, guaiol and/or beta-caryophyllene in a biosynthetic process.

As already mentioned, the invention also relates to a pharmaceutical formulation comprising the plant extract, preferably, Cannabis plant extract as obtainable by the methods described herein. The pharmaceutical formulation may have the chemical composition of the Cannabis plant extract as obtainable by the methods described herein, or may contain the Cannabis plant extract as obtainable by the methods described herein and further substances, including, but not limited to, carriers, surface-active agents, thickeners, adjuvants and the like in any suitable concentration. The solvent as described herein, may be completely or partially removed prior to incorporation of the Cannabis plant extract into the pharmaceutical formulation, e.g. by heating the extract under reduced pressure (e.g. under vacuum). The skilled person is aware that some volatile plant metabolites may be removed with the solvent. Alternatively, the solvent may be included in the pharmaceutical formulation as a carrier.

The term “pharmaceutical formulation” as used herein generally defines a formulation suitable for application/administration to the body to treat, care for or improve the appearance of the body. The pharmaceutical formulation of the invention is specifically intended to be applied/administered to a subject which is a patient, preferably a human patient. But it is also envisaged that animals, in particular companion animals are treated with the herein described Cannabis plant extracts. The term “pharmaceutical formulation” can be used interchangeably with “medicament”.

It is envisaged that the herein described compositions or pharmaceutical formulation are for use in medicine.

Likewise, it is envisaged that the plant extract, preferably, Cannabis plant (soft) extract as obtainable by the methods described herein is for use in medicine. Accordingly, the invention relates to a Cannabis plant (soft) extract as obtainable by the methods described herein for use in medicine.

In other words, the invention relates to a method of treatment comprising administering a Cannabis plant (soft) extract as obtainable by the methods described herein to a subject in need thereof.

The pharmaceutical formulation described herein may be used in the treatment and/or prevention of a condition and/or disease associated with pain.

In particular, the invention relates to a Cannabis plant (soft) extract as obtainable by the methods described herein for use in the treatment and/or prevention cancer pain, acute non-cancer pain, chronic non-cancer pain and/or complex pain syndromes, more specifically chronic cancer pain, somatic pain, visceral pain, central neuropathic pain, peripheral neuropathic pain and/or complex pain syndromes. A specific example of cancer pain would be chronic cancer pain. Specific examples of acute non-cancer pain are somatic pain including pain caused by/resulting from tooth extraction, minor cutaneous surgery, skeletal trauma, orthopedic surgery or tension headaches, and visceral pain including pain caused by/resulting from dysmenorrhea, acute pancreatitis or renal/biliary colic. A specific example of chronic non-cancer pain is neuropathic pain such as central neuropathic pain including post-stroke thalamic pain or pain caused by/resulting from spinal cord injury, and peripheral neuropathic pain including pain caused by/resulting from post herpetic neuralgia, diabetic painful neuropathy, trigeminal neuralgia, idiopathic small fiber polyneuropathy or antiretroviral therapy-induced neuropathy. Specific examples of complex pain syndromes include fibromyalgia syndrome, complex regional pain syndrome and migraine.

In other words, the invention relates to a method of treatment of cancer pain, acute non-cancer pain, chronic non-cancer pain and/or complex pain syndromes, more specifically chronic cancer pain, somatic pain, visceral pain, central neuropathic pain, peripheral neuropathic pain and/or complex pain syndromes comprising administering a Cannabis plant extract as obtainable by the methods described herein to a subject in need thereof.

As used herein, the terms “treating”, “treatment” and the like are understood as affecting a subject, tissue or cell to obtain a desired pharmacological and/or physiological effect in terms of a partial or complete cure of a disease or associated symptoms. The terms “preventing”, “prevention” and the like are understood as prophylactic treatment of the subject in terms of completely or partially preventing the occurrence, arresting the development or reducing the severity of a disease or associated symptoms. The term “subject” as used herein refers to a mammal, preferably a human.

Such methods and uses comprise administering to a patient in need thereof an effective amount of the Cannabis plant extract, formulation or pharmaceutical formulation described herein. The term “effective amount” is understood as an amount sufficient that when administered to the patient, the drug is provided to achieve the desired effect. A “therapeutically effective amount” may be determined by the treating physician.

The Cannabis plant extract, the composition or pharmaceutical formulation as disclosed herein may be administered locally or systematically. It may be administered by any suitable means, including oral, oromucosal, rectal, nasal, topical (including dermal, buccal and sub-lingual), vaginal or parenteral (including intramuscular, subcutaneous and intravenous) administration or in a form suitable for administration by inhalation or insufflation. It is preferred that the pharmaceutical formulation as disclosed herein is administered by oral, oromucosal or topical administration as well as inhalation. It is most preferred that the pharmaceutical formulation as disclosed herein is administered by oral administration.

The Cannabis plant extract as obtainable by the methods described herein may be further processed by any means known in the art to obtain a composition and/or pharmaceutical formulation that is suitable for administration to an animal or human. In other words, the pharmaceutical formulation may be prepared by any means known in the art. In yet other words, the pharmaceutical formulation may be prepared by any means known in the art from the Cannabis plant extract as obtainable by the methods described herein. It may be prepared as solids, such as tablets or filled capsules, or liquids such as solutions, suspensions, emulsions, elixirs, or capsules filled with the same, all for oral use, in the form of suppositories for rectal administration, or in the form of sterile injectable solutions for parenteral (including subcutaneous) use. Formulations for parenteral administration include sterile aqueous or non-aqueous solutions, suspensions and emulsions.

The skilled person will understand that the pharmaceutical formulation as described herein is formulated so as to be suitable for application to a patient, to be compatible with the pharmaceutical active substances present in the composition and to not cause any unreasonable safety or toxicity concerns. The pharmaceutical formulation described herein can be administered to the subject at a suitable dose. The dosage regimen will be determined by the attending physician and clinical factors.

The present invention is also illustrated by the appended non-limiting FIGURE and Examples.

### Examples

The following Examples illustrate the invention

### 1. Cultivation of the Cannabis Plant Material

Cultivation took place in a closed greenhouse which was supplied with filtered air via an HVAC system. To avoid the spread of diseases, beneficial insects were used in the cultivation and plants were regularly visually checked for diseases by trained gardeners. Cannabis plants can be propagated by taking cuttings from mother plants. Therefore, mother plants were held as the stock for the production plants. Cuttings taken from the mother plants were sticked in rockwool cubes and forced to root for 3 weeks at temperatures for about 25 to 29° C. and a high air humidity (typically about 70 to 90% relative humidity). After rooting the plants entered into the vegetative growth phase, where the plants grew bigger for about 2 weeks at temperatures of about 22 to 28° C. and a relative humidity of about 60 to 80%. Once the plants were big enough and ready for flowering, the flowering phase was initiated by reducing the hours of light from 18 h a day to 12 h a day. At the end of flowering (after 8 to 9 weeks) the female flowers were harvested from the stems. The stem was cut at the lower part and the flowers were separated from the stem. The separated flowers were trimmed and dried in a drying chamber for 96 hours at 26° C. by constantly removing humid air.

| | | Mother | Vegetative | Flowering |
|---|---|---|---|---|
| Parameter | Cuttings | plants | Phase | phase |
| Air - Temperature | 25-29 | 22-28 | 22-28 | 22-28 |
| [° C.] | | | | |
| Air - Humidity [%] | 70-90 | 60-80 | 60-80 | 30-50 |
| Light - Day Length | 18 | 18 | 18 | 12 |
| [hours] | | | | |
| Light - Intensity | about 200 | about 600 | 300-500 | about 550 |
| [μmol/m²/sec] | | | | |

### 2. Extraction of Dried Flowers

The flowers were trimmed prior to the drying step and had a minimum content of delta-9-THC equivalents (delta-9-THC+delta9-THCA*0.877) of 10 percent by weight. An amount of 16.7 kg (15.9 kg-17.5 kg) of ethanol 96% (v/v) of pharmaceutical grade (Kraul & Wilkening u. Stelling GmbH) was used per kg dried flowers. The extraction process was performed by exclusion of light at 15° C. to 25° C. in a stainless-steel extraction vessel (custom made by Edelstahlbau Tannroda GmbH) of cylindric shape and of pharmaceutical grade equipped with a strainer at the bottom. The extraction was subdivided in maceration (static extraction) and percolation (mobile extraction). The dried flowers were first soaked with half the amount of ethanol (8.35 kg±5% per kg flowers) for 24 h±1 h during maceration which enabled to dissolve most of delta-9-THC and other cannabinoids as well as related secondary plant substances like terpenes (aroma), fats and chlorophylls. After maceration, the second half amount of ethanol was added to the miscella, and the percolation was started. Thereby, the miscella of ethanol and Cannabis extract was seeped through the flower material with a flow rate of 4.0 to 5.0 ml/min per kg flowers for 69 to 86 h and collected in a mobile buffer tank made of pharmaceutical stainless-steel (custom made by Edelstahlbau Tannroda GmbH).

### 3. Filtration of Percolate

Smaller flowers residues that have not been separated with the strainer of the extraction vessel were removed via filtration of the percolate through a polypropylene-based particle filter (Pall GmbH, order no. AB2A0157PH4) of pharmaceutical grade and a deposition rate of 1.5 μm. The filter was inserted into a stainless-steel housing and the percolate was pumped through the filter with a peristaltic pump and collected in a stainless-steel tank. The operating pressure and temperature should not exceed 5 bar and 50° C., respectively.

### 4. Concentration of Percolate

The extraction solvent was evaporated in vacuo by using a rotary evaporator (Manufacturer: Büchi; Model: Rotavapor R-220 Pro; Distillation rate: up to 12 liters ethanol/h; Flask size: 20 liters; Maximum capacity of the flask: 12 liters; Continuous feed). The water bath temperature, the pressure, and the rotation speed amounted to 72° C., 185 mbar and 150 rpm, respectively. These conditions led to a steam temperature of ethanol of 41° C.-42° C. and an evaporation performance of approximately 10 L/h. The evaporation was concluded when the steam temperature dropped below 30° C. indicating the almost full removal of ethanol. An almost solvent-free soft extract of dark green to dark brown resin-type nature was obtained mainly consisting of delta-9-THCA and partially activated delta-9-THC.

### 5. Decarboxylation of Solvent-Free Soft Extract

The full transformation of delta-9-THCA to delta-9-THC was conducted via thermally induced decarboxylation in vacuo. Therefore, the rotary evaporator was used by adjusting the water bath temperature, pressure, and rotation to 80° C., 185 mbar and 150 rpm, respectively. The decarboxylation time amounted to 72 h. The resulting soft extract still had a dark green to dark brown resin-type nature with an average delta-9-THC content of 70 to 74 percent by weight of the extract. The conversion factor between dried flowers and soft extracts amounted to 4.7 on average.

### 6. Analysis of the Obtained Extract

The Cannabis plant soft extract was visually inspected to define the color which was yellow to green.

The extract was further analysed via TLC according to Ph.Eur. 2.2.27 and according to DAB monograph “Eingestellter Cannabisextrakt”. The extract was further analysed via HPLC according to Ph.Eur. 2.2.29 and according to internal validated method on the basis of the DAB monograph “Eingestellter Cannabisextrakt” by comparing the cannabinoids profile with retention times of reference standards and by a reference chromatogram of this product.

The extract was further analysed via GC according to Ph.Eur. 2.2.28 and according to internal validated GC-MS method by comparing the terpenes/terpenoids profile with retention times of reference standards and by a reference chromatogram of this product.

The water content was determined according to Ph.Eur. 2.5.12 (Karl Fischer) and the ethanol content was analysed according to Ph.Eur. 2.2.28 and according to internal validated GC method. Microbial impurities were analysed according to Ph.Eur. 5.1.4-2 including Ph.Eur. 2.6.12 and 2.6.31.

The result of the analysis of the obtained extract is shown in FIG. 1 and Tables 1 to 7.

### 7. Analysis of the Obtained Extract Regarding Waxes

1.00 kg soft extract was mixed with 1.75 kg of ethanol and stirred for 30 minutes. The obtained solution was filtered through a 71 μm metal mesh sieve and Ethanol was added to obtain a soft extract to ethanol solution with a ratio of 1:2.3. The solution was placed in a freezer for 48 hour (winterization). After 48 hours there was no significant fatty layer on the top, only a few small streaks of opaque plaques/strings were visible. Filtration using a 50 μm and second 21 μm metal mesh sieve was performed. A dark fatty material was collected. The collected dark fatty material was solved in about 30 ml ethanol and centrifuged. Approximately 20 grams of wax was obtained. Which means that the wax content was about 2%.

1. A method for producing a Cannabis plant extract comprising delta-9-tetrahydrocannabinol (THC) from a Cannabis plant comprising the following steps:
(a) providing a Cannabis plant which comprises the THC in a sufficient amount;
(b) trimming and drying the flower material separated from the remaining plant material; and
(c) treating the flower material of step (b) with a solvent and separating the Cannabis plant extract comprising the THC from the flower material.

2. The method of claim 1, wherein the method further comprises evaporating the solvent from the liquid Cannabis plant extract to concentrate the extract.

3. The method of claim 2, wherein the method further comprises heating the concentrated Cannabis plant extract to a temperature of 50° C. to 150° C. to decarboxylate tetrahydrocannabinolic acid (THCA) to THC.

4. The method of claim 1, wherein the Cannabis plant comprises the THC in an amount of at least 1 percent by dry weight (w/w).

5. The method of claim 1, wherein the drying of the flower material of the Cannabis plant in step (b) is performed at a temperature range of 20° C. to 35° C. until the water content of the flower material is below 10 percent.

6. The method of claim 1, wherein the treating of the flower material in step (c) comprises the following steps:
(i) overlaying the flower material with the solvent in a ratio of about 1:8.4 (w/w) thereby obtaining a mixture of the flower material and the solvent;
(ii) performing maceration for about 24 h at a temperature of 15° C. to 25° C. and exclusion of light;
(iii) adding further solvent thereby obtaining a mixture of the flower material and solvent in a ratio of about 1:16.8 (w/w);
(iv) performing percolation, wherein the solvent is seeped through the flower material with a flow rate of 3.55 to 5.45 ml/min per kg flower material for 63 to 97 h at a temperature of 15° C. to 25° C. and exclusion of light; and
(v) separating the Cannabis plant extract from the Cannabis flower material.

7. The method of claim 6, wherein the separating of the Cannabis plant extract in step (v) comprises filtering the Cannabis plant extract with a deposition rate of 1.5 μm.

8. The method of claim 1, wherein the solvent is selected from the group consisting of ethanol, butanol, alkanes, ethyl ether, tert butyl-methyl-ether, methyl-ethyl-ketone, acetone, ethyl acetate and CO₂.

9. The method of claim 1, wherein the method does not involve a winterization step.

10. A Cannabis plant extract comprising the THC as obtainable by the method of claim 1.

11. The Cannabis plant extract of claim 10, wherein the plant extract is in liquid form.

12. The Cannabis plant extract of claim 10, wherein the plant extract is in solvent-free and decarboxylated form.

13. The Cannabis plant extract of claim 10, wherein the Cannabis plant extract comprises delta-9-tetrahydrocannabinol (THC) in an amount of at least about 60 percent by weight of the extract.

14. The Cannabis plant extract of claim 10, wherein the Cannabis plant extract further comprises one or more terpene(s) selected from the group consisting of alpha-bisabolol, guaiol, and beta-caryophyllene.

15-16. (canceled)

17. A method of treatment and/or prevention of chronic cancer pain, somatic pain, visceral pain, central neuropathic pain, peripheral neuropathic pain or complex pain syndromes, the method comprising administering the Cannabis plant extract of claim 10 the subject.

18. The method of claim 8, wherein the alkanes is selected from the group consisting of pentane, heptane, and propane.

19. A Cannabis plant extract comprising the THC as obtainable by the method of claim 2, wherein the Cannabis plant extract comprises the THC in an amount of at least about 60 percent by weight of the extract.
